(19) United States
(12) Patent Application Publication
(10) Pub. No.: US 2025/0341750 A1
(43) Pub. Date: Nov. 6, 2025

(54) Large-scale electrophysiology amplification platform (LEAP)

(71) Applicant: The Board of Trustees of the Leland Stanford Junior University, Stanford, CA (US)

(72) Inventors: Yi Shiou Duh, Stanford, CA (US); Mark L. Brongersma, Menlo Prk, CA (US); Hongquan Li, Stanford, CA (US); Siddharth Doshi, Stanford, CA (US); Martin Hrton, Bilovice nad Svitavou (CZ); Ching-Ting Tsai, Stanford, CA (US)

(21) Appl. No.: 19/197,011

(22) Filed: May 2, 2025

Related U.S. Application Data
(60) Provisional application No. 63/641,711, filed on May 2, 2024.

(51) Int. Cl. G02F 1/1516; A61B 5/00; G02F 1/157

(52) U.S. Cl. CPC G02F 1/15165; A61B 5/0075; G02F 1/157; A61B 2562/0285; G02F 2201/302; G02F 2203/10; G02F 2203/15

(57) ABSTRACT

A nano-photonic chip for optical readout of biological activity is provided. The design has an electrochromic polymer layer (e.g. ProDOT) on top of a conductive path layer. The electrochromic polymer layer is capable of changing optical properties of the electrochromic polymer layer in response to the biological activity. A nearfield optical resonator is formed between the conductive path layer and the electrochromic polymer layer, where the incoming light, which has approximately normal incident to an optical substrate, is transmitted through the optical substrate and coupled by the conductive path layer into the nearfield optical resonator. Nearfield confinement enhances absorption in the electrochromic polymer layer, and then changes in refractive index encode the biological activity and modulate the optical properties of outgoing reflected or transmitted light for optical readout therewith facilitating the optical readout of the biological activity.

### CROSS-REFERENCE TO RELATED APPLICATIONS

This application claims priority from U.S. Provisional Patent Application 63/641,711 filed May 2, 2024, which is incorporated herein by reference.

### FIELD OF THE INVENTION

This invention relates to devices, methods and systems for monitoring electrophysiological activity.

### BACKGROUND OF THE INVENTION

Monitoring electrical activity of neuron and heart cells has greatly advanced our understanding of electrophysiological dynamics of neurons and cardiac tissues. Understanding the coordinated neural activity underlying brain function requires the simultaneous monitoring of large numbers of individual neurons.

Microelectrode arrays (MEAs) with highly multiplexed recording sites, such as the Neuropixels, have demonstrated the ability to record ˜1,000 neurons simultaneously in a mouse brain. However, the necessity of a wired input/output (I/O) connection poses a major obstacle to increasing the number of recording sites without significantly enlarging the implant's footprint. Additionally, the fixed electrode positions in MEAs restrict recordings to predefined brain regions.

In addition to MEAs, optical imaging of neural activity using genetically encoded voltage indicators (GEVIs) enables wireless, high-throughput recording from thousands of neurons. However, the requirement for genetic modification presents a significant challenge for clinical applications. Moreover, optical GEVI-based methods are limited by photobleaching, short recording durations, slow temporal resolution, and potential permanent alterations to cell membranes.

Therefore, there is a critical need for a scalable neural recording platform with high spatiotemporal resolution, high multiplexity, and a high density of recording sites, all without requiring genetic modification or fluorescence labeling.

### SUMMARY OF THE INVENTION

A conceptually new approach, LEAP, is provided herein to large-scale, label-free optical imaging of neural activity that harnesses the unique optical properties of nanopatterned meta-surfaces. Compared to conventional neural recording methods, this approach eliminates electrical wiring and bulky I/O interfaces for higher scalability. Furthermore, unlike existing optical neuroimaging methods based on GEVIs, LEAP is label-free and transgene-free, thus offering the potential for clinical translation. Therefore, the successful implementation of this innovation will transform the conventional approach of neural recording by introducing a highly-scalable, high-resolution, wireless, and label-free platform for both in vitro and in vivo neural recordings.

LEAP is designed to visualize the electrophysiological activity of neurons and cardiomyocytes. It has several aspects, including (1) the LEAP system, (2) a nanofabricated photonic chip, (3) a method to use the nanofabricated photonic chip, (4) a method to use LEAP, and (5) a manufacturing method for the nanofabricated photonic chip.

In an exemplary embodiment the invention can be characterized as a LEAP system with the following components:
A nanofabricated photonic chip.
A light illumination path to achieve a resonant condition of the nanofabricated photonic chip. One example of resonance condition is the near plane wave illumination with single wavelength. The illumination source can be laser, LED, SLED and redirected using beam splitter. The plane wave illumination can be achieved using lenses and beam splitter. The incident angle can be a normal incident to the chip. An optional polarization control mechanism may be applied to condition incident light, including a polarizer and polarization maintenance fiber.
A light readout path to monitor and map reflection readout of the chip. This path includes an objective lens and a tube lens to relay images from the sample plane to the camera plane. A Grin lens can also relay images from two conjugate planes.
A recording unit device, such as a fast-speed camera or an event-based camera, to capture reflection readout from the chip.

According to the exemplary embodiment, the nanofabricated photonic chip system has the following elements:
A sensing material with electrochromic properties, such as pi conjugated polymer material ProDot, p(g2T-TT). Those materials change their optical properties in response to a neural firing event. Take conjugated polymers as an example of the sensing material with two unique properties.
   (1) Polymer with a narrow and strong optical absorption peak due to the delocalized π bond that responds to local ion concentration perturbed by neuron activity. This intense absorption (i.e. ProDot around 550 nm) leads to a significant increase of its refractive index (RI) to 1.7 with low optical loss in the 550-800 nm range, as dictated by the Kramers-Kronig relations. The perturbation of this absorption causes the strong refractive index modulation to be further encoded into optical signal.
   (2) The oligoether side chains of polymers are highly hydrophilic, thus facilitating the intercalation of water molecules and ions in these side chains. Consequently, changes in the local electric field can induce ionic flow within these hydrophilic side chains. This ionic flow, in turn, affects the optical transition of the delocalized π bonds, leading to a significant change in the absorption peak at 550 nm and in the RI in the 550-800 nm range.
A photonic resonator that transduces refractive index change into measurable optical signals. The photonic resonance mode of the metasurface is highly confined in the polymer layer and enables sensitive detection of even slight RI variations in polymer by measuring its reflection transmission or scattering signal, thus facilitating optical readout of minuscule local electric field changes. The photonic resonator can be made of:
   (1) Periodic metal or polymer waveguide. Because the polymer with the previous property (1) has high RI and low absorption, a guided mode resonance condition confines the electromagnetic field directly inside the sensing material. For systems involving metal grating, this periodic metal grating also radiates lattice resonance to maximize light-matter interaction. Those resonances usually have a higher quality factor (narrow in spectrum)
   (2) Surface plasma mode from metal nanostructure. Surface plasma confines the photonic resonance mode in the boundary between metal and electrochromic material. This resonance usually has lower quality factor (is broader in spectrum). The surface plasma can be on top of the periodic metal strip or on the side wall of the metal nanostructure.
   (3) Hybridized mode of (1) & (2). When these two modes interact, destructive interference or critical coupling appear, which maximizes absorption and maximizes the electromagnetic field overlap within the sensing material. This strong overlap and critical coupling condition makes the system sensitive to perturbation from the ion or electric field.
A metal nanostructure, such as subwavelength periodicity grating made of gold or silver that resonates with the incident light. Metal nanostructure serves two roles.
   (1) The purpose of resonance is to optimize the overlap of electromagnetic field within the sensing material where RI changes. For example, the surface plasma mode and lattice resonance maximize light-matter interaction.
   (2) Another role of the metal grating is to couple normal incident light into the photonic resonator such as a polymer waveguide mode or a surface plasmonic mode to propagate along the sensing material. This enables normal incident light to couple and concentrate into the photonic resonator. The grating's ability to probe and collect with normal incident light makes the sensor chip compact.
An optional electronic large conductor that facilitates electron transport from a distance. This conductor can be used to (1) change the doping level of the sensing material and (2) maintain local charge neutrality by supplying electron or electron holes from a distance away such as in the biological medium. This ionic-electronic coupling balances ion flow from the neuron by migrating holes to balance and change the sensing material's color. For systems using metal nanostructure, the metal itself can be used as a large conductor.
An optional substrate material such as (SiO2, MgF2, water, biological liquid, and other low index material) that enhances the photonic mode. For example, low refractive index material can act as a cladding material to concentrate the photonic mode inside the high RI polymer sensing material. The substrate can be rigid or flexible.

In another exemplary embodiment the invention can be characterized as a method to use the nanofabricated photonic chip, including the following steps:
Precondition the illuminated light with angle (i.e. degree of collimation), polarization (s or p) and near normal incident into the chip.
Metal grating coupe light into the resonator so the electromagnetic field is highly confined in the resonator.
The polymer senses the ion flow within its hydrophilic side chain, which facilitate electron hole doping to changes polymer's optical properties in response to a neural firing event. At the location closer to source, polymers change color more than at the locations far away.
The light inside the resonator interacts with the changing-colored-polymer and encodes the color change into measurable optical signal such as reflective signals, or transmission and scattering signals.
The optical signals are collected using a microscope connected to a fast speed camera.
Optional spike sorting algorithm clean the data and showcase electrical signal of neuron.

In yet another exemplary embodiment the invention can be characterized as a method using LEAP including the following steps:
Position the nanofabricated photonic device in the near proximity of the target sample, such as neurons or cultured cells, to allow the sample's action potential to induce the ionic flow nearby the sensing material. The ionic flow affects the optical property of the sensing material.
Illuminate the nanofabricated photonic chip with pre-conditioned illumination to make the incident light resonate with nanofabricated devices in order to maximize the sensitivity, for example by using normally incident light as illumination path or using oblique-angle incident light using a prism or TIRF objective.
Designed nanopatterns enable incident light to couple from a normal angle into the metal-polymer grating. On resonance, the light field is highly confined within the sensing polymer.
The light encodes the optical property of the sensing material, and nanophotonic design facilitates optical readout of minuscule local electric field changes. The interaction involves localized resonators such as surface plasmon resonance coming from the metal grating to ensure high spatial resolution.
Optionally, suppress the background reflective signal through the interference within the nanostructure under critical coupling conditions or using Fourier optics. This would reduce the background signal to avoid camera saturation.
Collect the reflective light from the nanofabricated chip via the readout path using objective and a tube lens.
Record the relay image with a camera such as a fast speed camera or an event-based camera and, if needed, highlight the spike by data processing such as spike sorting or subtraction of neighbor values.

In yet another exemplary embodiment the invention can be characterized as a manufacturing method for the nanofabricated neuron ‘listener’ involving the following steps:
Nanofabrication Au or Ag gratings of target widths and period conditions on the low refractive index substrate using methods like e-beam lithography, photolithography, laser interference lithography, displacement Talbot lithography, nanoimprint lithography. And transform resist (such as PMMA) pattern using etching/metal liftoff using resist.
Spin-coat the sensing material like conductive polymer with a higher refractive index on target wavelength onto the gratings.
Prepare a measurement system such as microscope or endoscope on conditions such as illumination path, readout path, polarization control and camera. Microscopes can be upright/inverse/endoscope.
Optionally, Inspect/calibrate nanopatterned structures resonance, for example, by inspecting them with a sweeping wavelength laser or apply voltage gating with a potentiostat to validate voltage sensitivity.
Optionally, electrochemically break in polymer by running cyclic voltammetry to precondition polymer to microstructure to facilitate ion mobility or initial doping level.
Integrate into a functional sensor after processes such as dicing, packaging, sterilization, and mounting to be compact and allow conformal contact with the targeted brain slice, culture cells, acute brain in vivo. Position a brain slice directly on top of the photonic chip or compress the in vivo brain tissue directly using the photonic chip. Optionally suction of fluid to ensure conformal contact. This method is non-transgenic and label-free.

In yet another exemplary embodiment the invention can be characterized a nano-photonic chip for optical readout of biological activity. The nano-photonic chip has an optical substrate transparent for incoming light, and a conductive path layer on top of the optical substrate. In one example, the conductive path layer is a metal layer. In one example, the metal layer is a nanopatterned metal grating. In another example, the metal layer is a metallic metasurface with a two-dimensional periodicity. In still another example, the metal layer is a metal grating with a dual-layer nanopatterned grating structure having a unit cell, such that the combined structure exhibits multiple photonic modes with differing symmetry, or is a metal grating with a dual-layer nanopatterned grating structure, wherein each layer containing at least one grating elements of differing geometry within a unit cell.

The metal layer serves both as a nanophotonic coupling structure and as a conductive layer that acts as a charge reservoir for the electrochromic polymer. This dual functionality is important to ensure rapid charge transfer and enable full electrochemical modulation within the timescale of neural activity (e.g., ˜1 ms). The polymer itself doesn't need to be highly conductive. What matters is that there's a conductive pathway to supply or extract charge from the polymer backbone. In the exemplary implementation provided herein, the metal layer happens to serve both as the optical resonator and the charge reservoir. Other conductive layers (e.g., ITO) could serve the same electrochemical function, even if they're not involved in the photonic coupling.

The nano-photonic chip further has an electrochromic polymer layer (e.g. ProDOT layer) on top of the conductive path layer. The electrochromic polymer layer is capable of changing optical properties of the electrochromic polymer layer in response to a biological activity. The electrochromic polymer layer has an optical absorption peak in a range of 400 nm to 700 nm, and is further characterized that optical absorption perturbation changes a refractive index of the electrochromic polymer layer.

The electrochromic polymer layer comprises a polymer backbone that supports a dominant π-π optical transition, and hydrophilic side chains that facilitate ionic interaction with a surrounding medium.

The electrochromic polymer layer exhibits a dominant π-π optical absorption peak between 500 nm and 650 nm, corresponding to its neutral-state transition prior to polaron formation.

The nano-photonic chip further a nearfield optical resonator formed between the conductive path layer and the electrochromic polymer layer. The incoming light transmitted through the optical substrate is coupled by the conductive path layer into the nearfield optical resonator, such that nearfield confinement enhances absorption in the electrochromic polymer layer, and the changes in refractive index encode the biological activity and modulate the optical properties of outgoing reflected or transmitted light for optical readout therewith facilitating the optical readout of the biological activity.

The incoming light is incident approximately normal to the optical substrate and outgoing light is reflected approximately normal to the optical substrate. The incoming light, incident approximately normal to the optical substrate surface, is coupled by the metallic layer into a near-field optical resonator formed between the metal layer and the conductive polymer. The near-field confinement within the resonator enhances the absorption response of the polymer to local electric field perturbations. The modulated optical properties of the polymer subsequently couple back into outgoing light, which exits the device at approximately normal incidence. This normal-incidence coupling and detection pathway distinguishes the system from scattering-based detection approaches, enabling efficient wide-field optical readout without requiring dark-field or oblique-angle illumination.

The near-field resonator is selected from the group consisting of a plasmonic resonator, lattice resonator, a guided-mode resonator, a Fabry-Pérot resonator, and a gap-mode plasmonic resonator.

The biological activity comprises electrical, electrochemical activity or a combination thereof, including but not limited to neural activity, retinal activity, cardiac activity, and skeletal or smooth muscle electrical signaling.

In a further embodiment, the nano-photonic chip could further have a conductive path that includes: (i) an ionic solution penetrating the electrochromic polymer layer to form a local ionic interface with the polymer backbone, and (ii) a connection to a metal layer positioned outside a region of primary refractive modulation, the metal layer maintained near the solution's equilibrium potential, and functioning as a charge reservoir to enable fast electrochemical modulation of the electrochromic polymer layer.

For this embodiment, the electrochromic polymer layer then has hydrophilic side chains capable of ionic flow in response to the biological activity to charge a capacitor formed by the backbone of the conductive polymer layer and the ionic solution.

In still another exemplary embodiment the invention can be characterized a nano-photonic chip for optical readout of biological activity with the following variations to the embodiments described supra. The nano-photonic chip has an optical substrate transparent for incoming light and a metal metasurface on top of the optical substrate The metal metasurface has multiple patterned layers, each having one or more grating sub-elements with different geometries within a unit cell, configured as either a one-dimensional metasurface grating or a two-dimensional metasurface array. The nano-photonic chip further has an electrochromic polymer layer on top of the metal metasurface, where the electrochromic polymer layer is capable of changing optical properties of the electrochromic polymer layer in response to a biological activity. A nearfield optical resonator formed between the metal metasurface and the electrochromic polymer layer, where the incoming light transmitted through the optical substrate is coupled by the metal metasurface into the nearfield optical resonator, and where the changes in refractive index encode the biological activity and therewith facilitating optical readout of the biological activity.

In this embodiment, the electrochromic polymer layer could exhibit a dominant π-optical absorption peak between 500 nm and 650 nm, corresponding to its neutral-state transition prior to polaron formation.

Embodiments of the invention are useful in, e.g. Drug Discovery Research, Neural Monitoring in Drug Trials, Wireless Intraoperative Monitoring, Epileptic Brain Tissue Localization, Neural Prostheses for Paralysis (Brain-Machine Interface), or Stroke Patient Recovery Monitoring with optical ECog.

Embodiments of the invention can have the following advantages:
1. Wireless and Free-Space Interface: LEAP introduces a fully wireless and free-space neural monitoring interface, eliminating the complexities associated with wiring and routing in traditional methods. This innovation significantly simplifies setup and enhances the mobility of neural monitoring systems. Competing technology: wired multi-electrode arrays (MEAs), patch clamp, High density CMOS array, wired electrocorticography (ECoG) all require wiring and amplifier and complex multiplexing mechanism. So less adaptable for acute monitoring during surgical procedures.
2. Compact Design: LEAP's compact design offers greater flexibility in integrating high-density neural monitoring into various medical and research settings, reducing the need for complex bulky neural probes and equipment. Other technology: Technologies include ECORE (ElectroChromic Optical Recording), where action potential induced changes in optical properties of a plain electrochromic thin film without any photonic nanostructures are monitored and CAGE (Critically coupled waveguide-Amplified Graphene Electric field imaging device), where changes in optical properties of graphene films are monitored and the “Electro-plasmonic nanoantenna”, where electrochromic changes in a polymer coating modify the resonance of individual resonant metal nanoparticles resulting in a change in scattered light intensity. Both ECORE, CAGE use bulky I/O interfaces such as prism. To compensate for the small intrinsic changes in optical properties of electrochromic films, ECORE only has one pixel recording because ECORE requires a complicated balanced detector. To operate with a degree of band filling such that its optical properties are sensitive to small changes in potential, CAGE requires application of a high gate voltage. Along with the limited spatial resolution, this restricts the utility of such a method in wirelessly measuring neural activity with high spatial resolution. The electro-plasmonic nanoantenna monitors signals stemming predominantly from localized resonances in individual metal nano-antennas. The intensity of light modulated by collections of these local metal resonators is small compared to the intensity of transmitted or reflected light that doesn't interact with the resonators. This necessitates the use of specialized dark field microscopes to isolated scattered signals and achieve a sufficient signal-to-noise ratio, significantly limiting broader uptake. The photonic design of the LEAP system, with embodiments such as the design of hybrid dual-mode nanostructured optical resonators with even and odd modes allows normally incident light to be coupled into the system and interfere to cancel reflected/transmitted light. Increasing the signal-to-noise by avoiding stray unmodulated light and allowing for coupling into the structures at normal incidence allows for compatibility with standard brightfield optical microscopys, enabling usage by a non-specialist microscope users and hence its commercial viability.
3. High-Density Single Unit Action Potential Readout: LEAP achieves high-density single-unit action potential readout, allowing for precise and detailed neural data collection, rather than low density EEG, ECog array for only brain wave and local field potential. This advancement provides superior resolution compared to existing methods. Competing technology: (ECoG, EEG) in vitro, MEA in vitro.
4. Label Fluorescence Dye Elimination so human compatible: LEAP eliminates the need for label fluorescence dye, simplifying neural monitoring processes and reducing potential side effects or invasiveness associated with such dyes. Competing technology: Fluorescence Dye-Based Monitoring like Calcium and voltage indicator used in rodents.

ProDot is an example of organic mixed ionic-electronic conductors:
1. Structure and Composition:
   Backbone: The polymer features a π-conjugated backbone which enables delocalization of π-electrons across the polymer chain. This backbone structure is critical for the electronic transport properties of the material.
   Side Chains: Attached to the conjugated backbone are hydrophilic side chains, such as alkoxy groups or ethylene glycol derivatives. These side chains enhance the solubility and ionic conductivity of the polymer, thereby facilitating easier penetration of ions.
2. Optical Properties:
   Ionic-Electronic Interaction: The interaction between ionic charges. (from external sources such as neurons or cardiomyocytes) and the polymer electronic transport results in a modulation of the electronic structure of the backbone. This modulation, often referred to as doping, alters the optical absorption characteristics of the polymer, particularly influencing π-π transitions and polaron states.
   Optical Absorption and Refractive Index: The change of the sharp, narrow optical absorption perturbs the real part of the refractive index. These perturbations are the highest where the optical absorption is off the peak, providing a sensitive mechanism for optical sensing. Take ProDot as an example, the π π transition is around 550 nm and 600 nm-850 nm is where the refractive index strongly perturbs and low optical absorption, described by Kramers-Kronig relations.
3. Examples of Polymer:
   Oligoether-functionalized Propylenedioxythiophene (ProDOT)
   p(g2T-TT): Poly(2-(3,3′-bis(2-(2-(2-methoxyethoxy)ethoxy)ethoxy)-[2,2′-bithiophen]-5-yl)thieno[3,2-b]thiophene), abbreviated as p(g2T-TT)

In still another embodiment, the present invention provides a method for detecting biological electrical activity using a nanophotonic metasurface coupled to an electrochromic polymer. The method is characterized by illuminating a metasurface device at or near normal incidence and detecting modulation in the reflected light at near normal angle. intensity due to refractive index changes in the electrochromic polymer layer induced by local electrical activity. Because the robust design of nanophotonics, it accepts a wider angle and wavelength, so not limit to laser source that produce speckel one would then be allowed to use broadband incoherent light source, such as an LED or lamp.

Unlike conventional techniques relying on oblique incidence or scattered light detection to boost their sensitivity, the metasurface is engineered to confine and amplify optical fields within the electrochromic polymer through resonant near-field coupling at the convenient normal incident. The resulting change in the polymer's refractive index alters the reflection characteristics of the device, which are measured by a camera or photodetector positioned along the normal reflection axis. This enables sensitive, wide-field optical recording of electrophysiological events such as neuronal spiking or cardiac action potentials without external coupling structures such as prisms.

In still other embodiments, the method can be further characterized by recording dynamic changes in reflected light intensity across an array of spatial locations on the device, enabling large-scale, label-free mapping of bioelectrical activity at high spatial and temporal resolution.

The system is configured such that incoming light is incident approximately normal to the optical substrate surface and is coupled by a metallic layer into a near-field optical resonator formed between the metal layer and the conductive polymer. The near-field confinement within the resonator enhances the absorption response of the polymer to local electric field perturbations. These perturbations modulate the polymer's optical properties, which subsequently affect the outgoing light that exits the device at approximately normal incidence.

This design enables efficient wide-field optical detection without requiring dark-field or oblique-angle illumination, distinguishing the system from scattering-based approaches. The near-field resonator overlaps both the metal layer and the conductive polymer, allowing strong interaction with the electrical activity in the polymer while maintaining a primarily planar resonator geometry with minimal volume.

Importantly, the interaction with the resonator not only enhances absorption within the conductive polymer but also modulates the reflected light, encoding the biological signal for optical detection. This mechanism enables sensitive readout of electrophysiological events, including but not limited to neural activity, retinal activity, cardiac activity, and skeletal or smooth muscle electrical signaling.

### BRIEF DESCRIPTION OF THE DRAWINGS

FIGS. 1A-E show according to exemplary embodiments of the invention a conceptual overview and design of LEAP for label-free optical recording of neural activity. (FIG. 1A) Schematic of LEAP: an optical microscope and a metasurface-coated polymer layer encode neuron electrical signals into light intensity changes, captured by a high-speed camera. Representative pixel traces show localized electrophysiological spikes. (FIG. 1B) Mechanism of neural activity encoding via modulation of ProDOT backbone charge density. (FIG. 1C) Optical response of ProDOT as a function of hole density. (FIG. 1D) Enhancement of signal sensitivity by underlying subwavelength gold (Au) grating metasurface. (FIG. 1E) Double-layer metasurface design to maximize light confinement and detection sensitivity.

FIGS. 2A-F show according to exemplary embodiments of the invention electrochemical and optical transduction mechanism of LEAP sensing. (FIG. 2A) Schematic of neuron-polymer ionic coupling, modeled as a capacitive interface. (FIG. 2B) Detailed model of current flow at the neuron-polymer interface based on Hodgkin-Huxley dynamics, highlighting ionic compensation pathways. (FIG. 2C) Influence of ProDOT thickness on charge density and voltage response; thinner films concentrate charge and enhance sensitivity. (FIG. 2D) Schematic of electrochemical setup and polaron state formation. (FIG. 2E) Optical absorption changes in ProDOT under varying backbone hole density, showing formation of polaron states. (FIG. 2F) Calculated refractive index modulation in ProDOT under applied voltage, according to Kramers-Kronig relations.

FIGS. 3A-H show according to exemplary embodiments of the invention nanophotonic resonator design for enhanced optical sensitivity. (FIG. 3A) Schematic of the dual-mode resonator concept enabling critical coupling via even and odd modes. (FIG. BA) Simulated sensitivity spectrum showing strong reflection modulation by either refractive index or absorption changes. (FIG. 3C) Reflection, transmission, and absorption spectra of the optimized structure under normal incidence. (FIG. 3D) Electric field distribution showing localized confinement at the corners of the gold strips. (FIG. 3E) Magnetic field enhancement due to circulating displacement currents in the trench regions. (FIGS. 3F-G) Electric field distributions illustrating symmetric electric dipole (ED) and antisymmetric electric quadrupole (EQ) contributions. (FIG. 3H) Multipolar decomposition confirming that the EQ mode aligns with high absorption and critical coupling conditions.

FIGS. 4A-H show according to exemplary embodiments of the invention array-based experimental validation of LEAP sensitivity. (FIG. 4A) Fabricated array of nanostructures with systematically varied top and bottom grating widths. (FIG. 4B) Wide-field optical image of the fabricated array after ProDOT coating. (FIG. 4C) Wide-field reflectance image at 660 nm, showing a continuous dark band corresponding to resonance-induced reflection dips. (FIG. 4D) Spatial map of reflection change across the array under square-wave voltage modulation between 0 mV and −100 mV. (FIG. 4E) Reflection signal traces from structures in array under square-wave stimulation. (FIG. 4F) Reflection percentage change (FIG. 4D normalized by FIG. 4C) from structures in array under square-wave stimulation. (FIG. 4G) Simulated reflection dark band matching with experimental results (FIG. 4H) Simulated reflection percentage change matching experimental results.

FIGS. 5A-D show according to exemplary embodiments of the invention large-scale label-free imaging of cardiac and neural activity using LEAP. (FIG. 5A) Spike waveform map of hiPSC-derived cardiomyocytes cultured on LEAP devices, overlaid on optical images. (FIG. 5B) Representative electrophysiological traces from two different locations, showing coordinated spontaneous action potentials. (FIG. 5C) Representative traces from multiple locations in an acute mouse brain slice placed directly onto LEAP without additional adhesion treatment. (FIG. 5D) Spiking activity emerges immediately following isoprenaline application, providing pharmacological validation of the bioelectrical origin of the recorded signals.

### LIST OF LABELS AND COMPONENTS

### Substrate and Layers

100—Optical substrate (mostly transparent substrate e.g. quartz or glass)
110—Nanopatterned metal grating (Bottom)
112—Unpatterned gold film
114—Conductive path layer
115—Top gold grating (for dual-layer structure)
130—Electrochromic polymer layer (e.g., ProDOT)
140—Nano-photonic chip (e.g. Metasurface 110+130) (110+130)
150—Nano-photonic chip as Dual-layer metasurface (110+115+130)

### Biological and Electrochemical Components.

200—Ionic solution Na+ and Cl− (e.g., saline or ACSF, share space with 130)
210—Neuron/Cardiomyocyte/Brain slice
240—Neural signal
250—Polymer side chain
260—Polymer backbone
270—Hole (positive charge carrier)

### Optical Pathways

300—Incoming light
320—Reflected light
330—Nearfield optical resonator
340—Direct optical path (reflection)
350—Resonant optical path (through near-field resonator)

### Optoelectronic Behavior

400—Working electrode (e.g., ITO contact)
410—Reference electrode (Ag/AgCl)
420—Counter electrode (Pt wire)
435—Capacitor formed between polymer backbone and ionic solution
450—π-π* absorption transition/peak
460—Polaronic absorption transition/peak
500—Camera or optical detector
501—Objective
502—LED or laser
503—Lens
504—Beam splitter
505—Camera pixel
520—Spectral region with large delta-k (optical loss)
530—Spectral region with large delta-n and low k (good for sensing)

### Photonic Modes

610—Even mode (symmetric photonic mode)
620—Odd mode (antisymmetric photonic mode)

### DETAILED DESCRIPTION

To address the challenges in the art, the inventors have developed a nanofabricated optical platform, termed the Large-scale Electrophysiological Amplification Platform (LEAP), designed for scalable, label-free optical imaging of neural activity. Compared to conventional Microelectrode arrays (MEAs), LEAP eliminates electrical wiring by using wireless optical detection, enabling high-density, large-scale recordings without bulky interfaces. Furthermore, unlike Genetically Encoded Voltage Indicator (GEVI) based optical methods, LEAP is entirely label-free and transgene-free, thus offering enhanced translational potential for clinical applications. Successful implementation of LEAP will transform the conventional approach to neural recording by introducing a highly scalable, high-resolution, wireless, and label-free platform applicable to both in vitro and in vivo systems.

### Overview of LEAP Sensing Mechanism

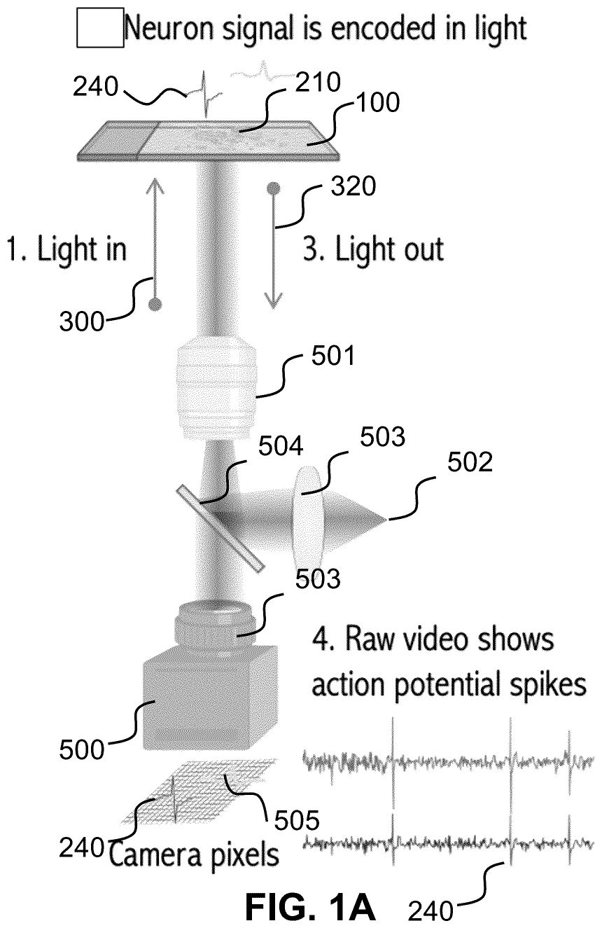

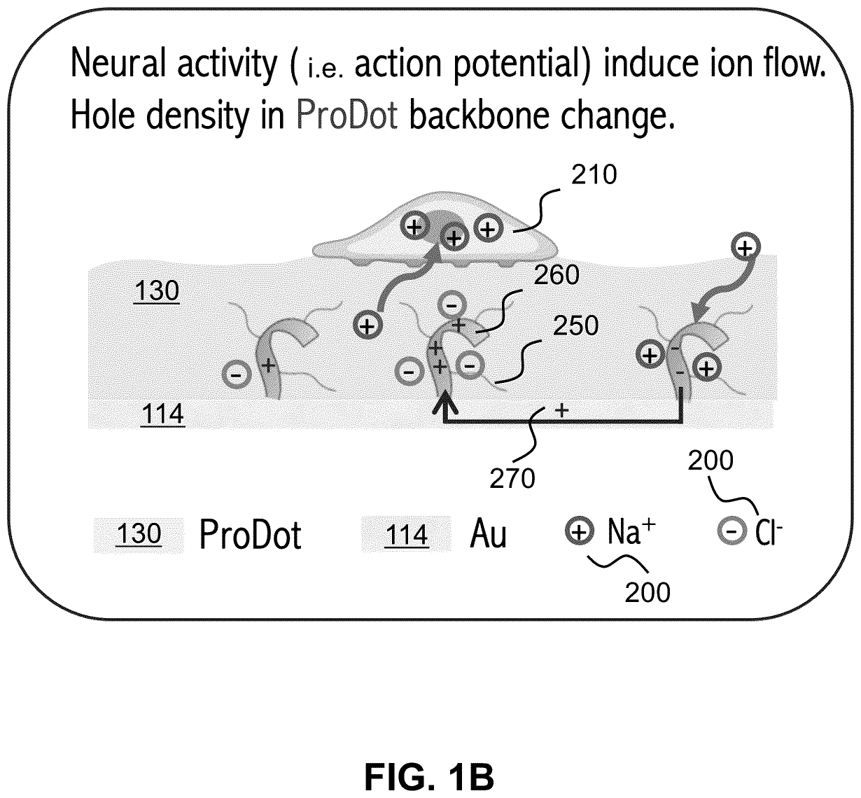

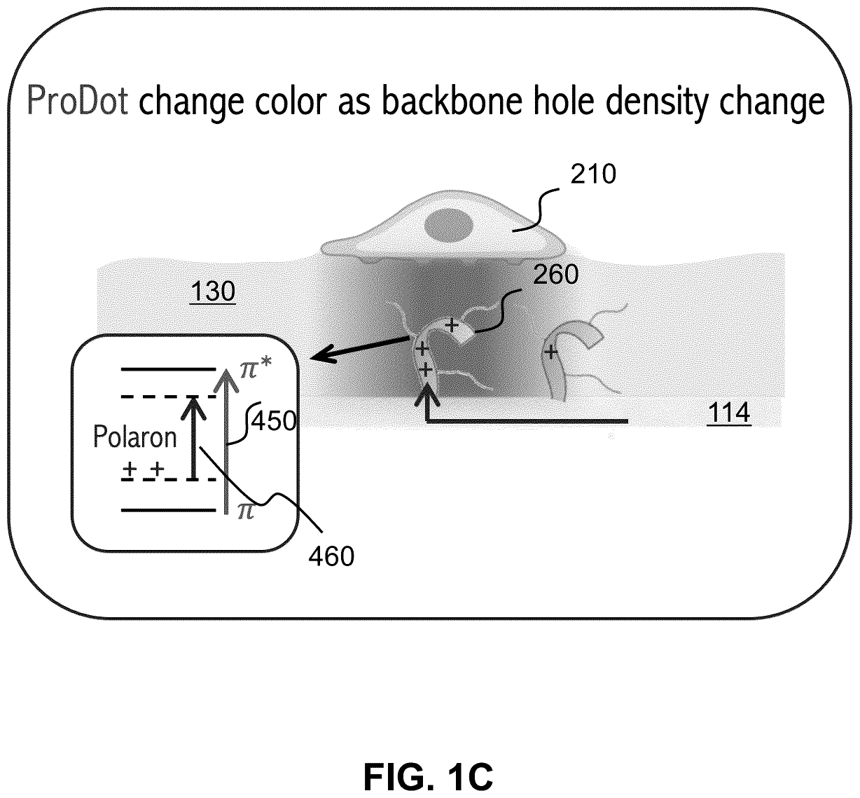

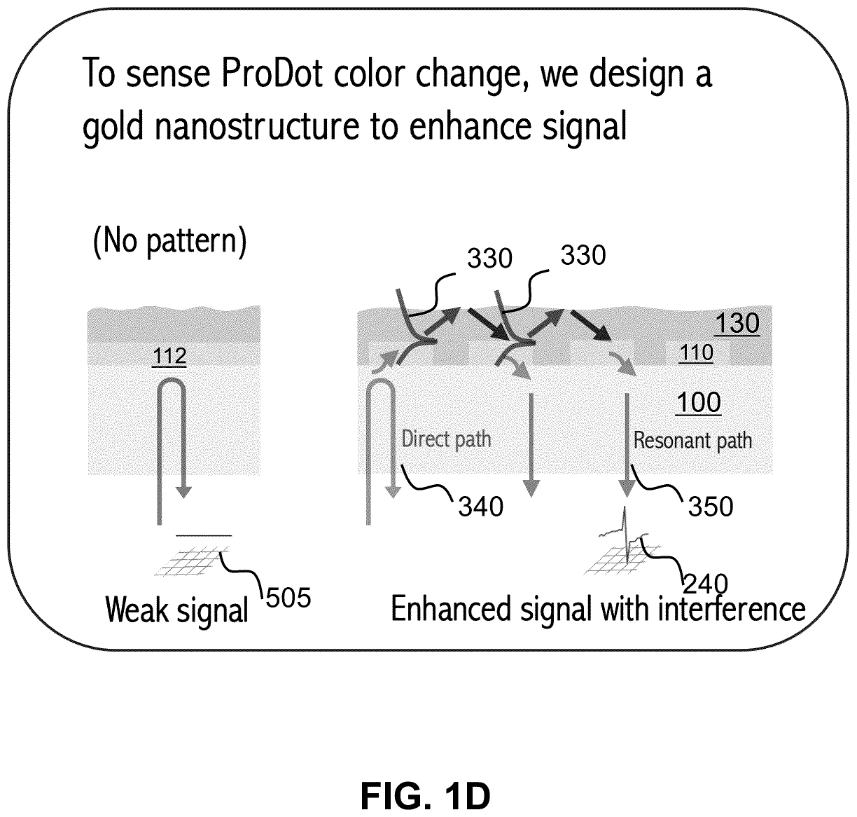

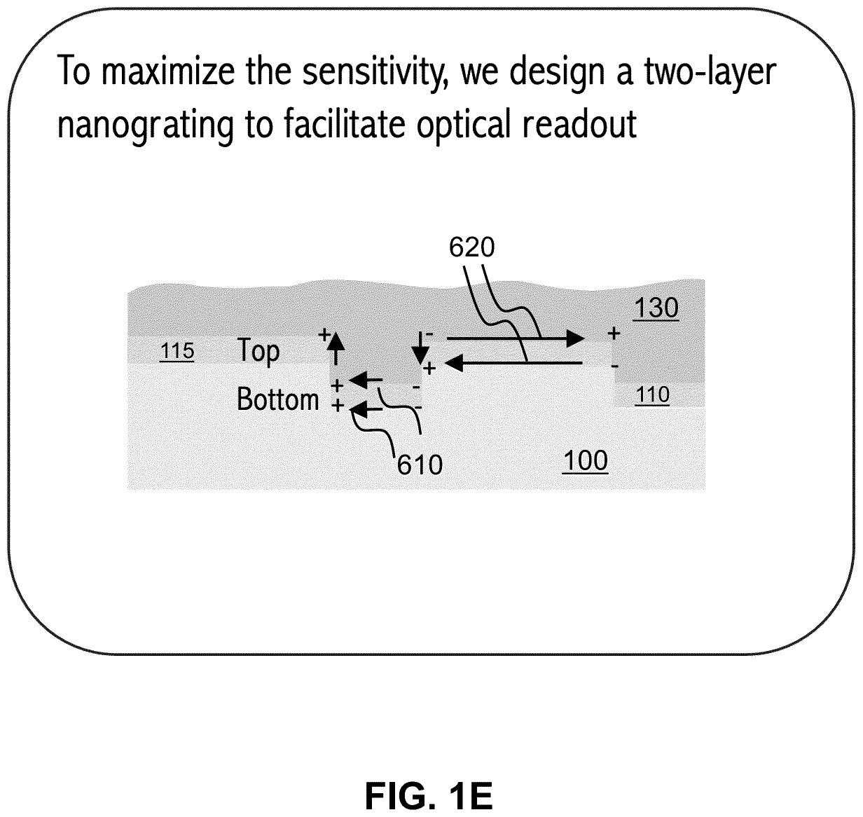

FIGS. 1A-E provide an overview of LEAP, illustrating how neural electrical activity is optically transduced by polymer film (130) and amplified by nanofabricated metasurface (140). In FIG. 1A, incoming light (300) illuminates the LEAP chip through a transparent substrate (100). The metasurface (140), which is coated with a thin electrochromic layer (130) of oligoether-functionalized propylenedioxythiophene (ProDOT), exhibits changes in the optical properties of the ProDOT layer in response to neuronal firing from electrogenic cells (210) such as cardiomyocytes and brain slice. Outgoing light (320), carrying the encoded neural signals (240), is captured by e.g. a camera (500) or video, with action potentials appearing as transient changes in pixel intensity.

The transduction mechanism relies on ionic-electronic coupling within ProDOT layer (130). As shown in FIG. 1B, when neurons fire action potentials, voltage-gated Na⁺ channels open, allowing Na⁺ ions to flow into the cells. Due to the hydrophilic oligoether side chains (250) of ProDOT (130), extracellular Na⁺ ions readily intercalate into polymer film (130). The departure of positive ions into the neuron leaves an excess of negative counter-ions (e.g., Cl⁻) within polymer film (130). To maintain local charge neutrality, positive charges (holes) are drawn into the polymer backbone (260) from the underlying gold conductive pathway (114), forming a capacitive double layer at the polymer-solution interface. As illustrated in FIG. 1B, the + symbols represent hole (270) accumulation within the polymer backbone (260).

This local modulation of hole (270) density strongly affects ProDOT's optical properties. In its neutral state, ProDOT exhibits a sharp π-π* absorption transition (450) centered around 550 nm. As hole (270) concentration increases, new polaronic absorption (460) states emerge, while the original π-π* transition diminishes, as illustrated in FIG. 1C inset. These electronic structure changes shift both the absorption and refractive index of the film, particularly enhancing refractive index tunability at longer wavelengths where optical loss remains low.

At the core of LEAP's amplification strategy is a nanopatterned gold metasurface (140) designed to magnify these subtle refractive index changes. ProDOT layer (130) is spin-coated atop a subwavelength gold grating fabricated on a SiO₂ substrate (100). Using rigorous coupled-wave analysis (RCWA), the inventors optimized the grating geometry to confine incoming (300) light tightly within ProDOT film (130), maximizing sensitivity to local refractive index perturbations.

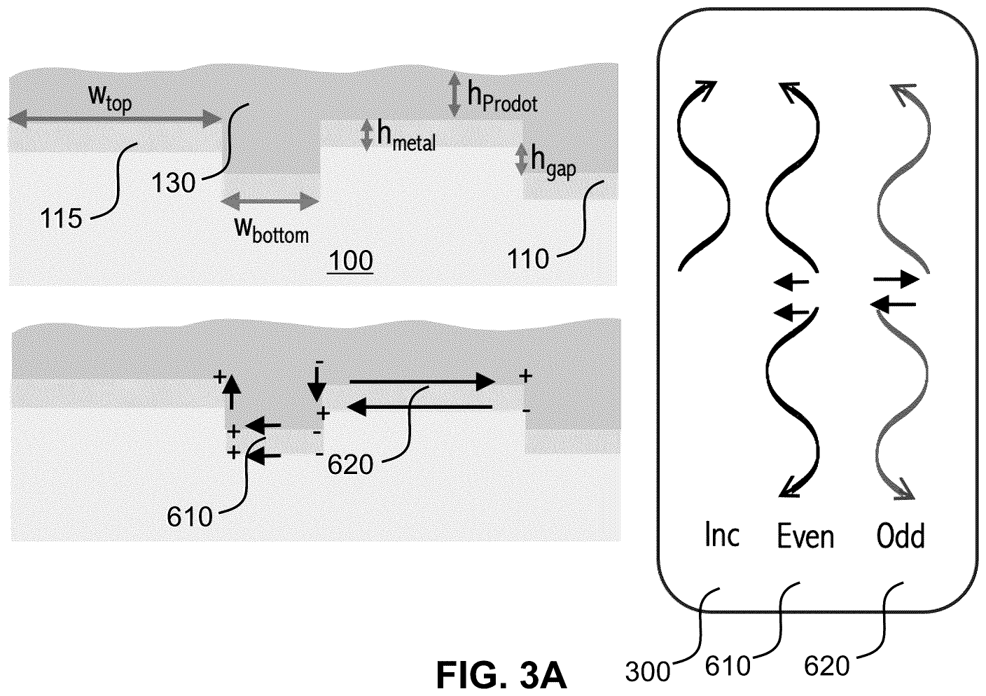

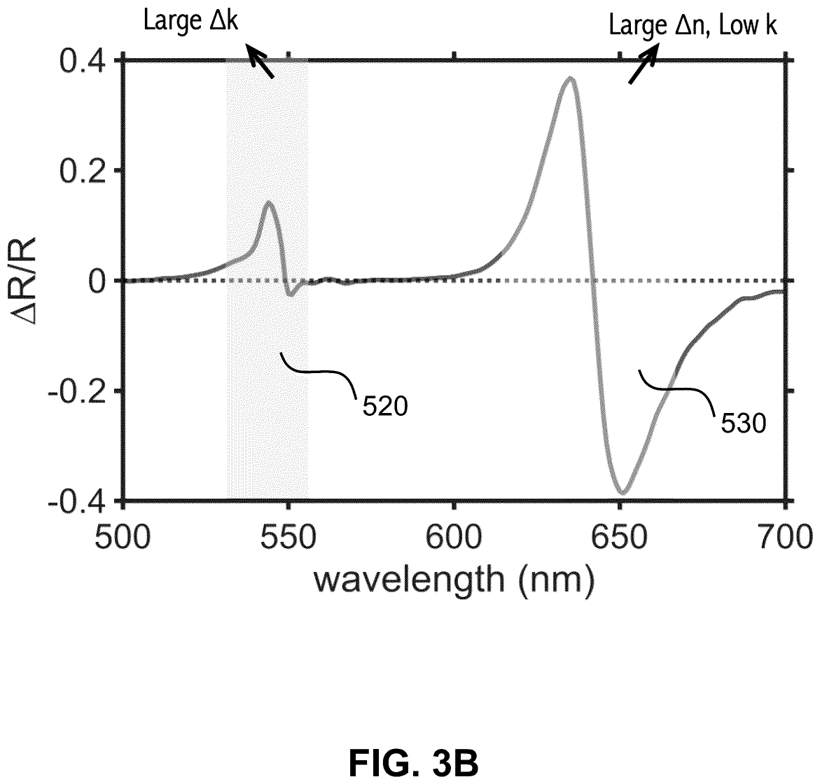

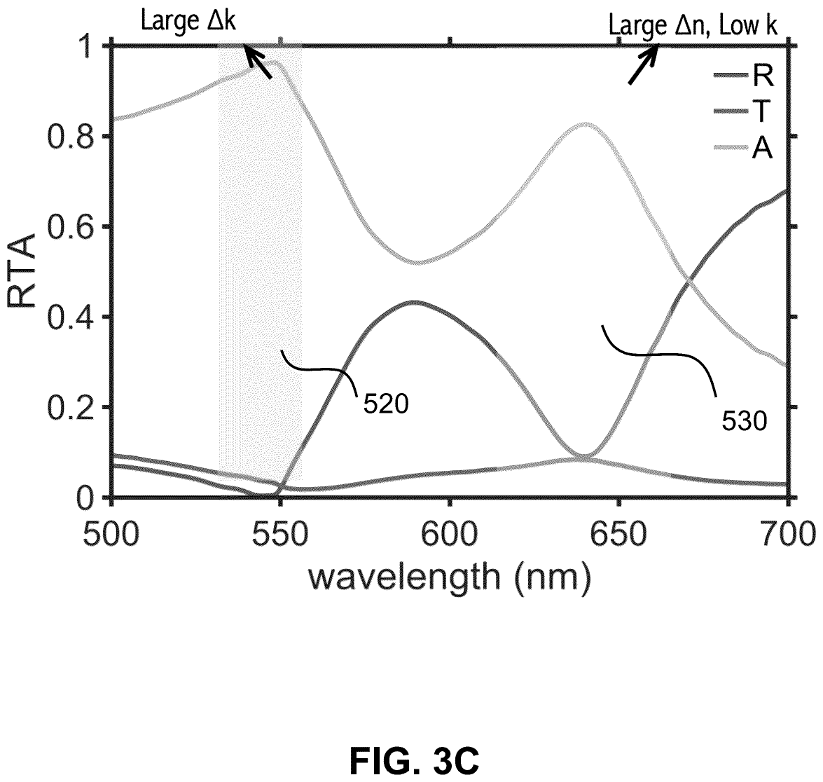

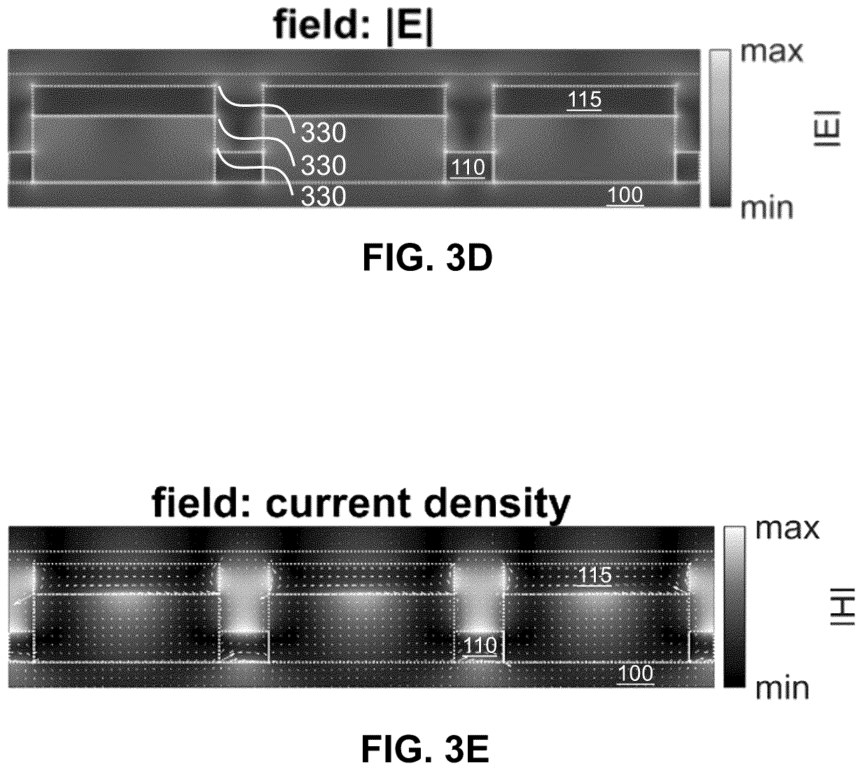

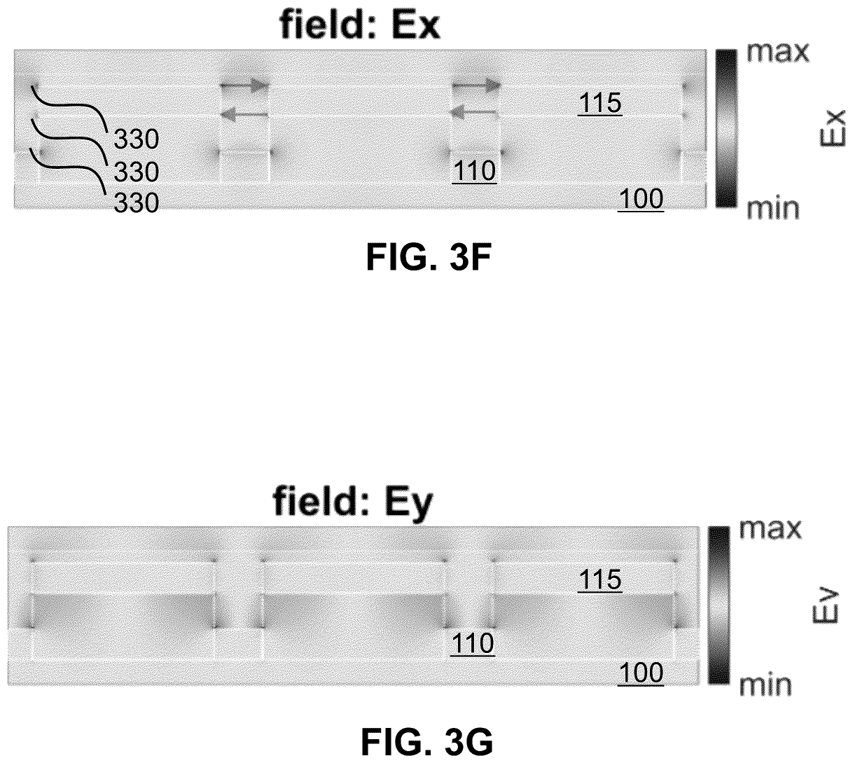

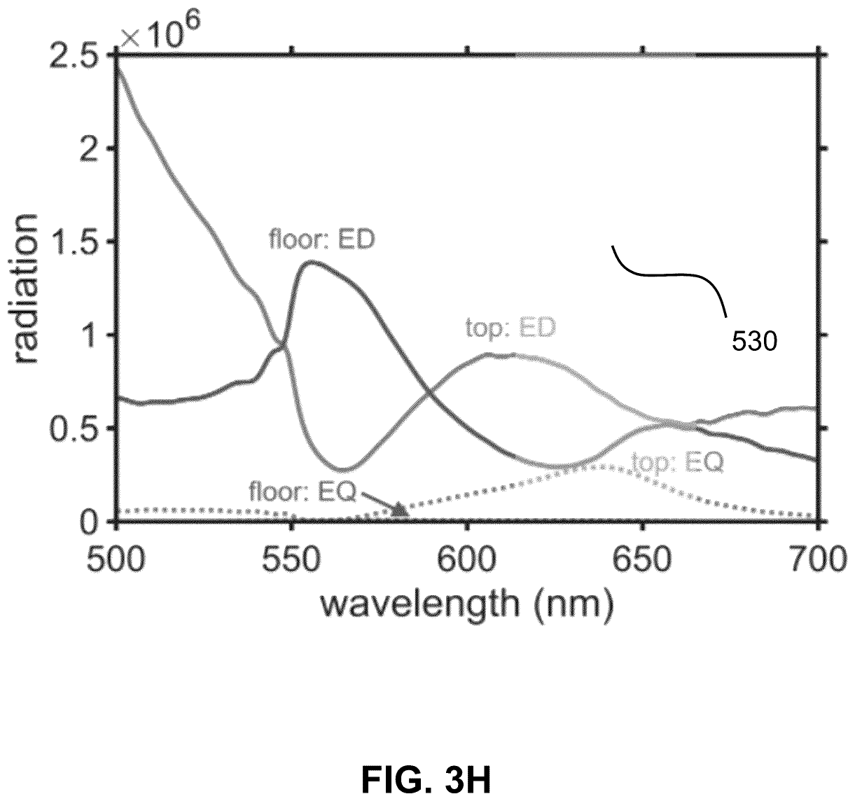

Metasurface (140) supports near-field surface plasmon resonances (330) that localize electromagnetic energy at the gold-polymer interface. As shown in FIG. 1D, unpatterned gold film (112) exhibits negligible optical modulation, whereas the nanopatterned metasurface (140) produces strong reflection contrast sufficient to resolve single-neuron action potentials. This pronounced reflection contrast arises from destructive interference between directly reflected light (direct path (340)) and the resonantly coupled light within the near-field resonator (330) (resonant path (350)). To further enhance sensitivity, the inventors also developed a double-layer gold grating structure (150) as shown in FIG. 1E, which will be discussed in detail in FIGS. 3A-H.

### Electrochemical and Optical Transduction Mechanism

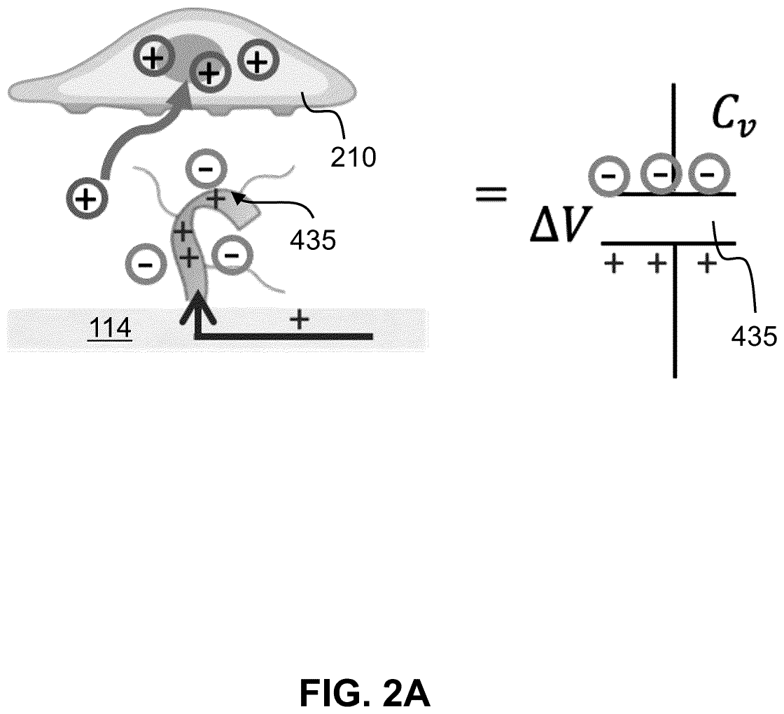

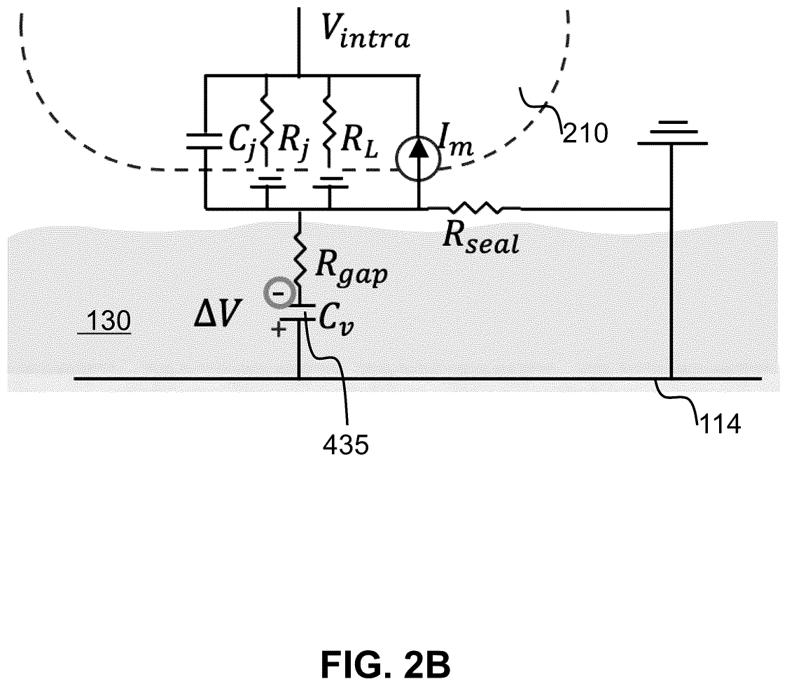

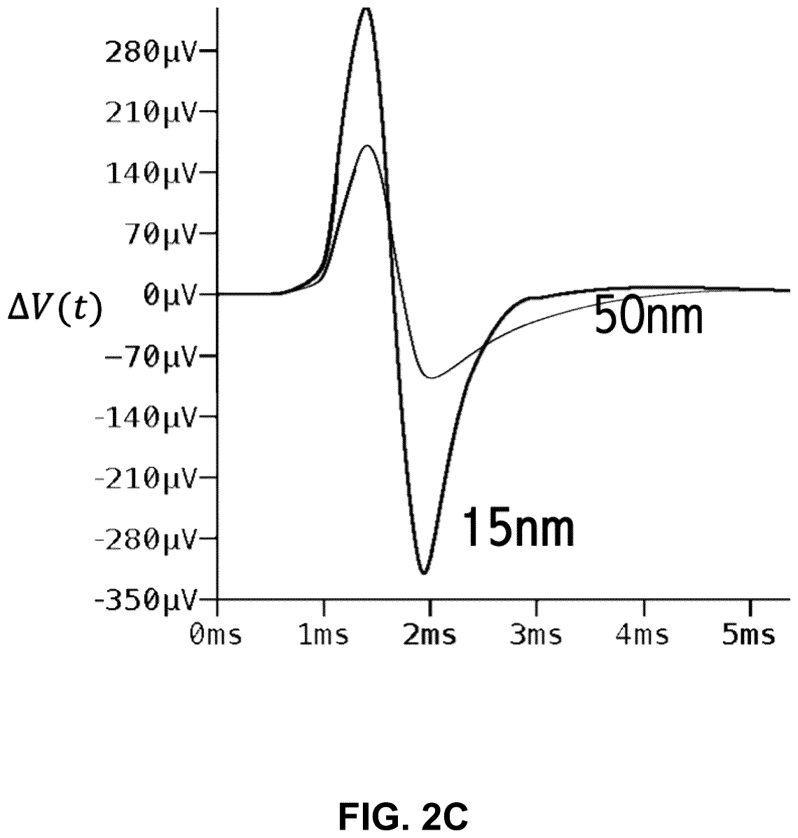

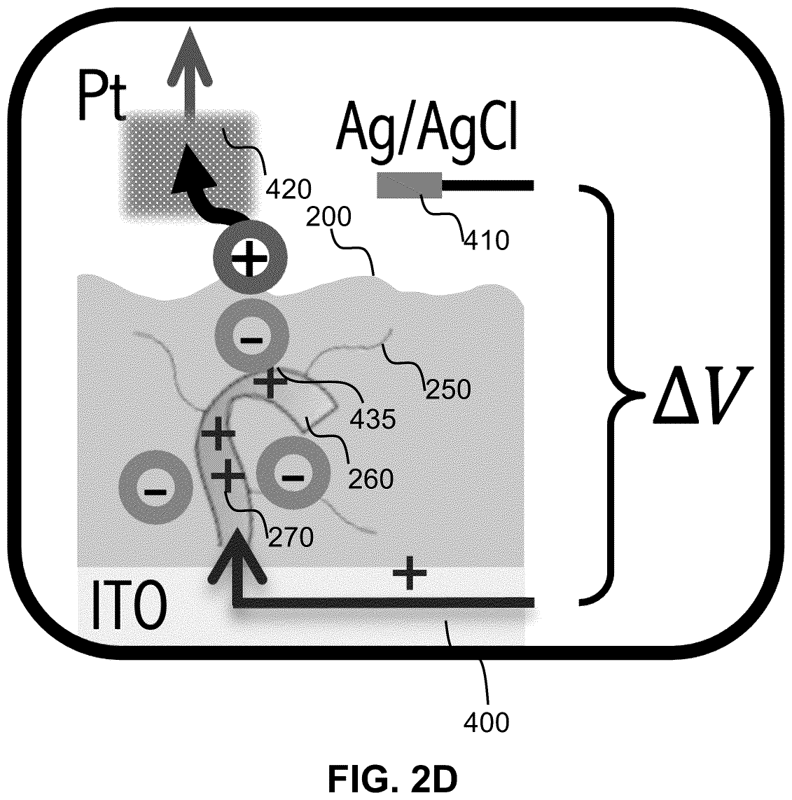

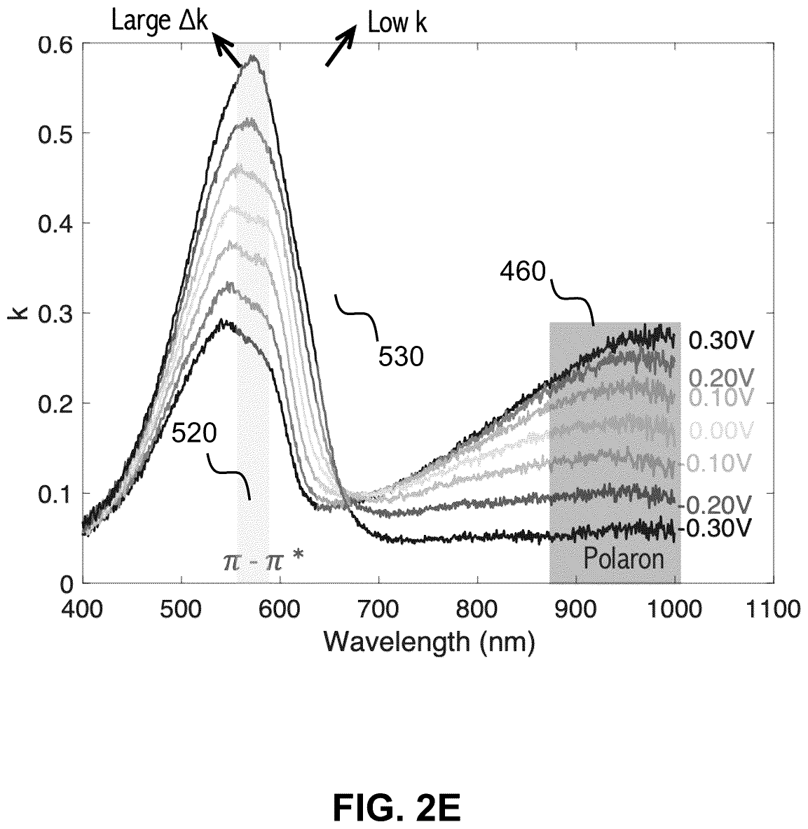

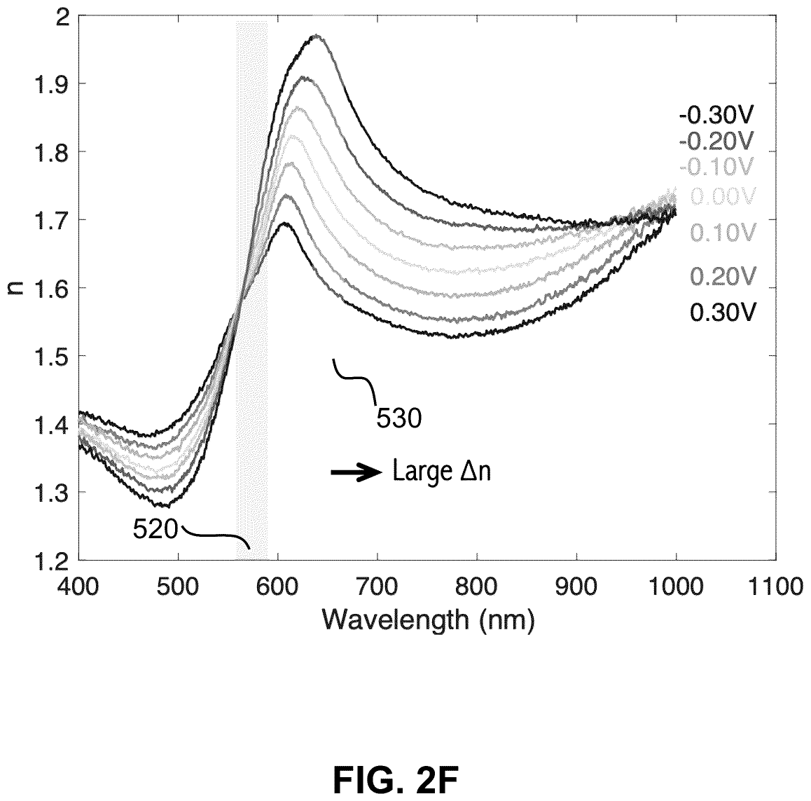

Ionic flow across the neuron-polymer interface can be modeled as charging a capacitor (435) formed between the ProDOT polymer backbone (260) and the surrounding ionic solution (200), as illustrated in FIG. 2A. The (−) charges in the ionic solution (200) and (+) charges (270) in the polymer backbone (260) represent the formation of a voltage difference (ΔV) across this capacitive interface (435). A more detailed model of this interface is shown in FIG. 2B, where ionic currents across the neuronal membrane are described using a Hodgkin-Huxley (HH) framework with membrane capacitance (Cⱼ), voltage-dependent ion channel resistances (Rⱼ) and a constant leak conductance (R_{L}).

When neurons fire, the net inward flow (Iₘ) of positive ions is compensated either by current leakage through the seal resistance Rₛₑₐₗ or by the extraction of positive charges (holes) (270) from the polymer's conductive backbone (260). Because ions cannot directly enter the π-conjugated polymer, and holes cannot transfer into the ionic solution (200), a capacitive interface (435) (with capacitance Cᵥ) naturally forms.

A critical aspect of LEAP's performance is the use of a high-electron-mobility gold conductive pathway (114), which acts as a rapid charge reservoir for the polymer film (130). This conductive pathway (114) enables full electrochromic switching of the ProDOT layer (130) within the short timescale of an action potential (˜1 ms), ensuring optical signals faithfully follow neural spiking dynamics.

The thin thickness of the polymer film (130) plays a critical role in optimizing its electro-optical sensitivity. ProDOT forms a volumetric network, and its total capacitance (435) scales with the film's volume. Since the ion flux generated by a single action potential remains roughly constant, thicker films dilute the induced charge density, whereas thinner films lead to a more concentrated modulation of charge density within the polymer backbone (260). As shown by the circuit simulation results in FIG. 2C corresponding to the model in FIG. 2B, thinner ProDOT layers exhibit higher volumetric charge densities and larger voltage drops (ΔV) across the polymer-solution interface, making electrical perturbations more readily detectable. Optimizing the photonic nearfield resonator (330) mode to be tightly confined within this thin ProDOT layer (130) further amplifies the optical response to subtle refractive index (RI) changes.

ProDOT effectively transduces hole (270) density changes into optical signals through its sensitive absorption characteristics. In its neutral state, ProDOT exhibits a strong π-π* (450) transition centered at 550 nm. As hole density increases, new polaronic absorption (460) states emerge within the π-π* (450) energy gap, leading to absorption at longer wavelengths while simultaneously suppressing the original transition, as depicted in FIG. 1C.

To experimentally validate this mechanism, the inventors applied bias voltages from −0.3 V to +0.3 V across the ProDOT film, gradually modulating the capacitive interface (435) using a three-electrode setup and a potentiostat as shown in FIG. 2D. The setup included a platinum (Pt) counter electrode (420), an Ag/AgCl reference electrode (410), and an indium tin oxide (ITO) working electrode (400). As shown in FIG. 2E, increasing the applied bias—and consequently the hole (270) density—leads to a reduction in the 550 nm π-π absorption (450) peak and an increase in absorption due to polaronic states (460) at longer wavelengths, consistent with FIG. 1C. Notably, the spectral region from 600-700 nm 530) exhibits low optical loss (low extinction coefficient k), while the 500-600 nm region (520), dominated by the π-π transition (450), shows strong absorption modulation.

According to the Kramers-Kronig relations, any change in optical absorption is accompanied by a corresponding change in refractive index. Specifically, enhanced absorption at 550 nm (520) spectral region leads to a significant increase in refractive index at longer wavelengths (650-700 nm) (530) while maintaining low optical loss, as illustrated in FIG. 2F. This optical window (530) enables the design of resonators that are highly sensitive to refractive index changes without incurring significant optical attenuation.

In summary, ProDOT exhibits several unique properties that make it particularly well-suited for LEAP. First, its hydrophilic oligoether side chains (250) promote the intercalation of water molecules and ions, enabling strong ionic coupling at the polymer-solution interface (435) and supporting tight adhesion with cultured cells. Second, its π-conjugated backbone (260) features a sharp and intense absorption peak (520) centered around 550 nm while remaining optically transparent beyond 650 nm (530). This combination of highly tunable absorption, low optical loss, and favorable electrochemical and interfacial properties makes ProDOT—and similar polymers—ideal materials for the optical transduction of bioelectric signals in LEAP.

Local electric field charges polymer backbone-solution capacitor (435). Next, ProDot transduce this hole (270) concentration change into optical readout.

### Nanophotonic Resonator Design for LEAP

To further enhance signal amplification and detection sensitivity, the inventors developed a two-layer gold nanograting structure (150) fabricated using electron-beam lithography. This dual-layer design substantially strengthens plasmonic nearfield resonator confinement (330), enabling highly sensitive optical detection of neuron-induced refractive index (RI) variations within the ProDOT film (130).

To maximize the optical response to small refractive index (RI) changes, the inventors aimed to confine the photonic resonance mode (330) entirely within the ˜50 nm ProDOT layer (130). Although thinner films enhance color modulation, achieving efficient optical confinement in such ultrathin layers did present challenges. A single symmetric dipole mode (610) cannot simultaneously cancel both reflection (320) and transmission, fundamentally limiting absorption to 50%.

To overcome this limit, the inventors introduced two photonic modes of opposite symmetry—even (610) and odd (620) modes. As illustrated in FIG. 3A (inset), incident light (300) destructively interferes with both modes. Under perfect destructive interference, no light escapes as reflection (320) or transmission, leading to full absorption—a phenomenon known as critical coupling. This configuration dramatically increases sensitivity to RI changes.

To realize this dual-mode condition, the inventors designed a two-layer coupled resonator (150) by stacking long (115) and short (110) metallic nanostructures on separate planes. This geometry creates trenches that drive circulating currents, generating an electric quadrupole (EQ) mode alongside the symmetric electric dipole (ED) response. The interplay between these modes critically enhances absorption efficiency.

The inventors performed systematic parameter sweeps using rigorous coupled-wave analysis (RCWA) to optimize the nanostructure for maximum reflection modulation. The most sensitive geometries were further evaluated using finite-element simulations (COMSOL Multiphysics). As shown in FIG. 3B, the optimized structure exhibits strong sensitivity across two main spectral regions, both for refractive index and absorption-based sensing. Notably, the sensitivity due to refractive index modulation is approximately four times greater than that from absorption changes, showing a unique advantage of our design.

Traditionally, enhancing absorption has been the primary strategy to strengthen light-matter interactions, reduce background reflection, and improve signal-to-noise ratios. Higher absorption allows greater illumination intensities without saturating the camera (500), given the increased relative reflection change (ΔR/R). However, for refractive index-based sensing, near-field confinement (330)—rather than simply maximizing absorption—is the critical factor.

Although absorption is slightly stronger near the polymer's π-π* absorption peak (450), optimizing for low-loss resonances ensures stronger local electric fields (FIG. 3D). In contrast, materials with high intrinsic absorption (520) damp the resonator fields, limiting sensitivity. Thus, the design strategy present herein prioritizes field confinement in low-loss materials (530) over maximizing bulk absorption.

The reflection dip observed in the optimized design directly results from strong near-field confinement (330) within the ProDOT layer (130). As shown in FIG. 3D, the corners of the gold strips localize intense electric fields, while the trenches enhance magnetic field confinement by inducing vertical current circulation.

Examining the field distributions in FIGS. 3F-G, the bottom gold layer (110) primarily supports a symmetric electric dipole (ED) mode, while the top gold layer (115) supports both a symmetric ED and an antisymmetric EQ component. Multipolar decomposition confirms this interpretation.

At the optimized wavelength, the optical response is governed by interference between:
the ED mode from the bottom layer (shifted by ˜¼ wavelength),
the ED mode from the top layer, and
the EQ mode from the top layer.

These multipoles destructively interfere in the farfield, suppressing radiative losses and maximizing absorption within the structure. Because this absorption occurs in a low-loss material (530), the resonator supports strong local electric fields. By perturbation theory, this high field confinement translates into large resonance shifts for small RI changes, yielding exceptionally high sensitivity.

### Array Fabrication and Experimental Validation

The design principles developed for LEAP extend beyond a single optimized geometry. A broad range of nanostructure configurations can achieve high absorption and high sensitivity. To demonstrate this generality, the inventors fabricated an array of structures by systematically varying the top (115) and bottom (110) gold grating widths. Fabrication details are provided in the Fabrication Method Section infra.

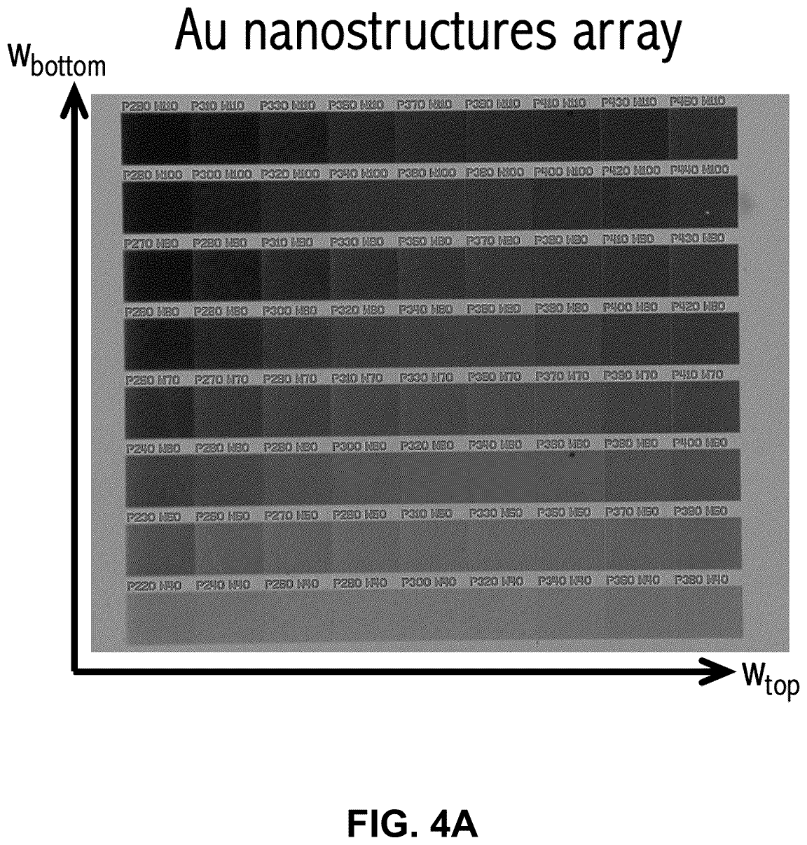

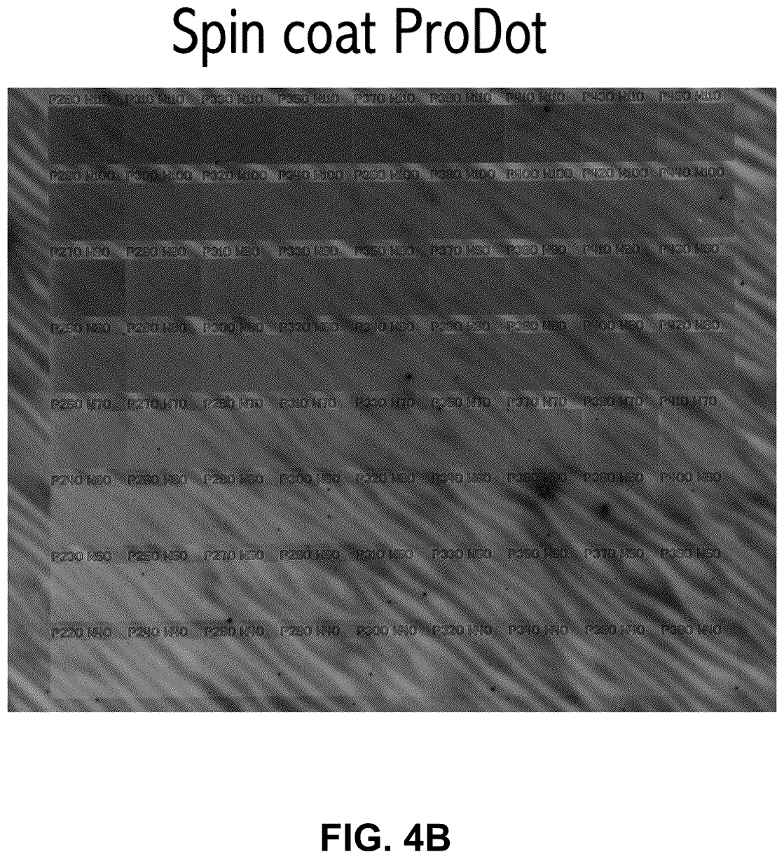

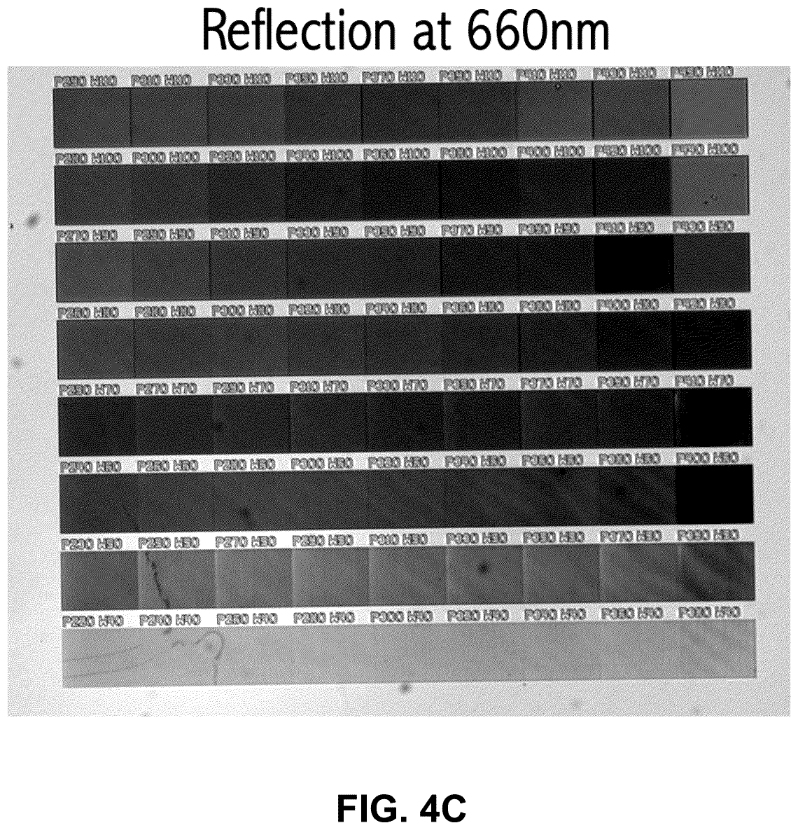

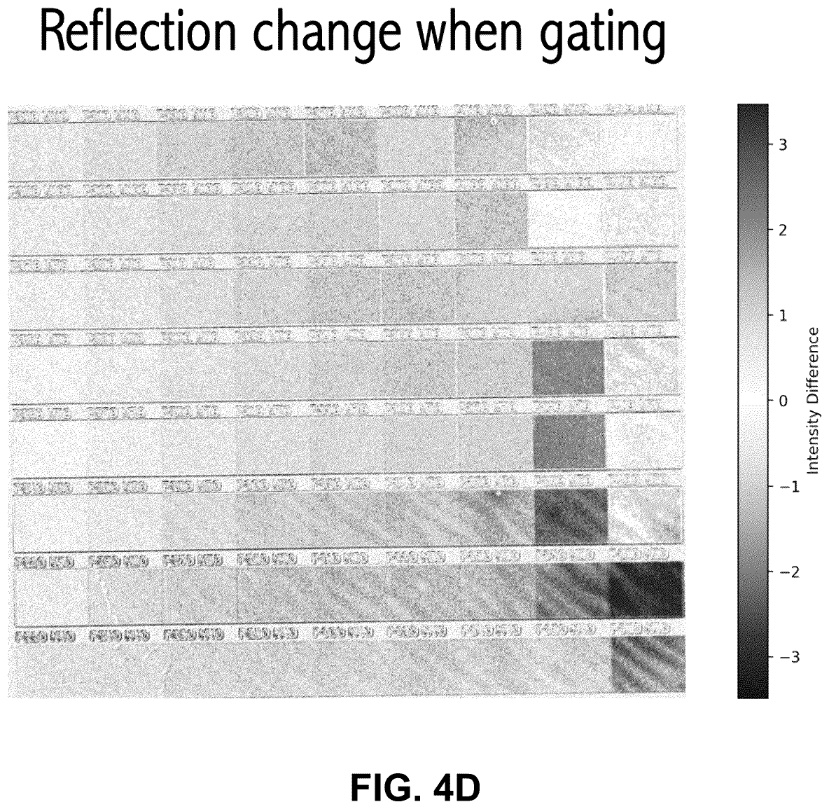

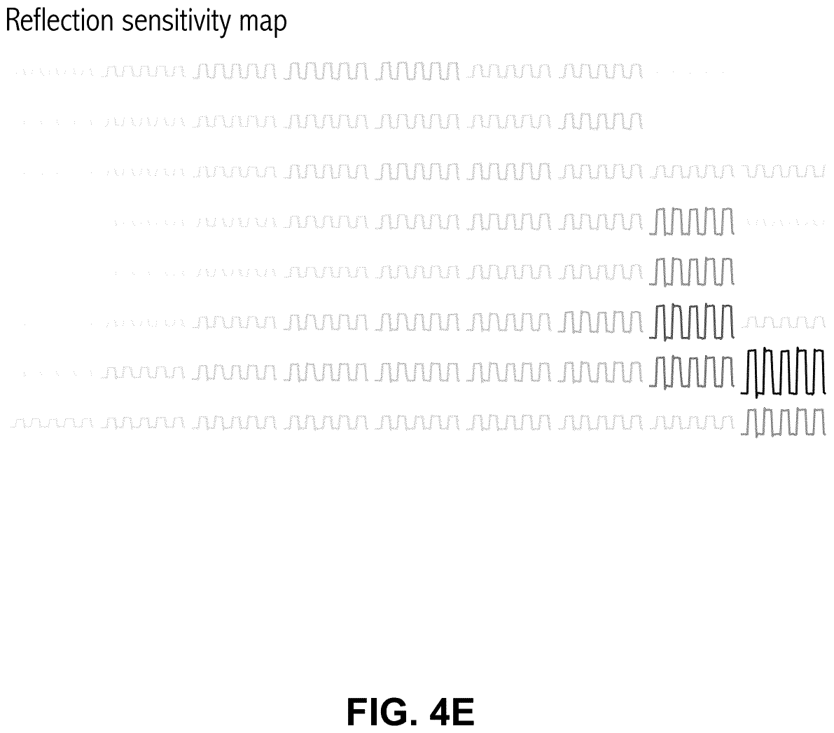

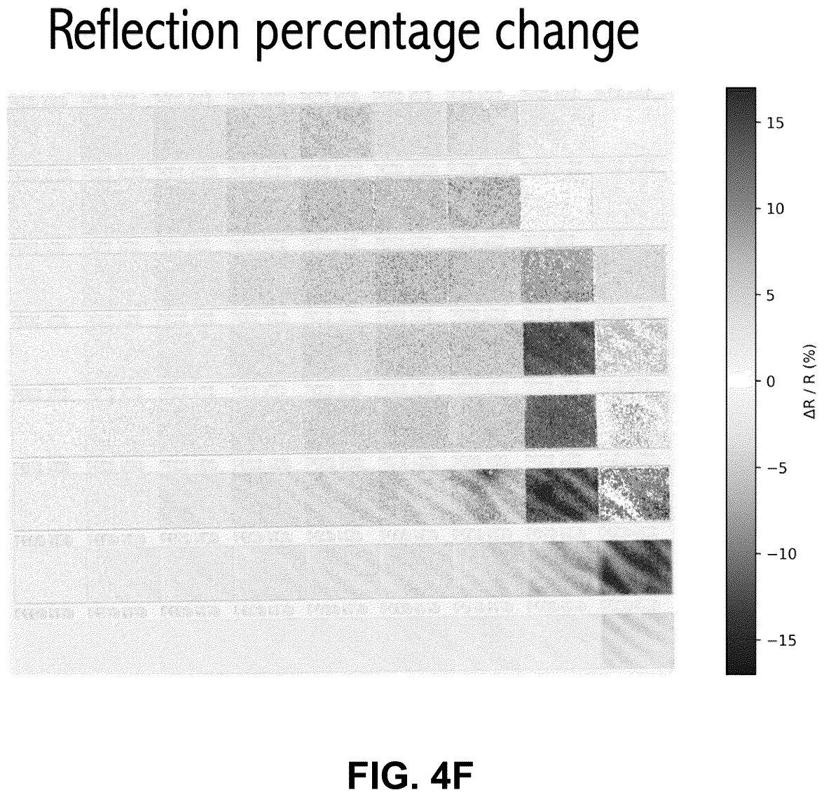

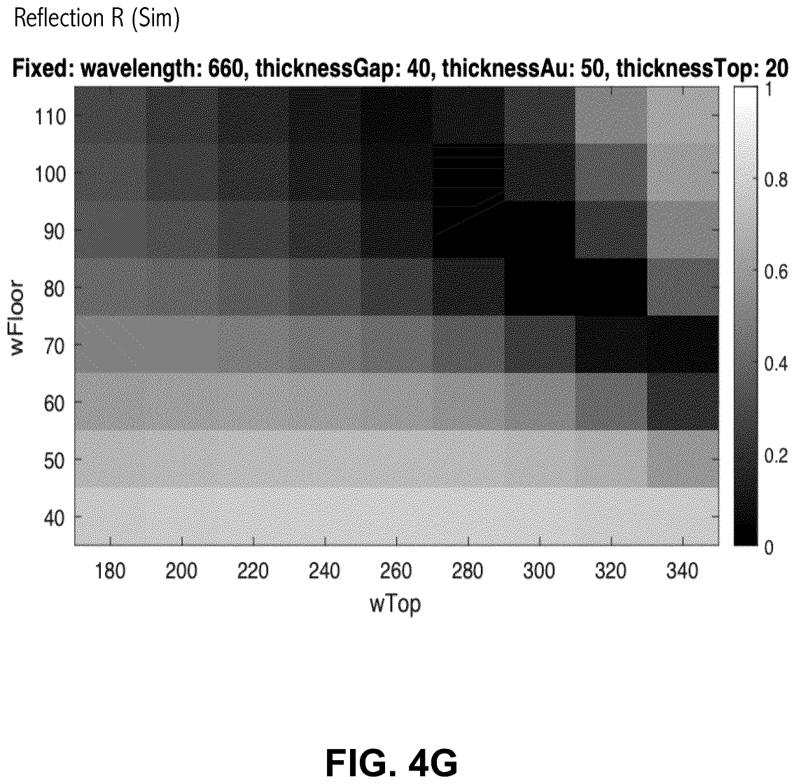

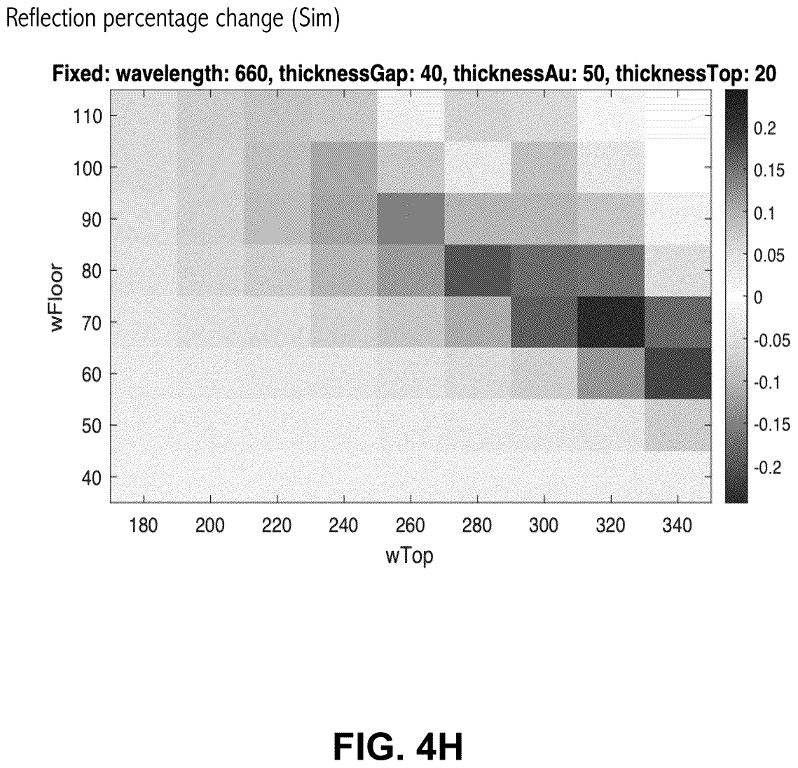

Each combination of top and bottom widths produces a distinct optical resonance, visible as different colors under white light illumination, as illustrated in FIG. 4A.

The image of LEAP chip after spin coat is as illustrated in FIG. 4B. The inventors illuminated the LEAP array using normally incident collimated wide-field 650 nm light (300) and simultaneously recorded the reflected signal (320) at the same wavelength at the normal angle with a high-speed camera (500). As shown in FIG. 4C, a continuous dark band appears across the array, corresponding to grating combinations that produce strong reflection dips. Based on the findings from FIGS. 3A-H, the inventors expect that structures exhibiting deeper reflection minima should correlate with higher optical sensitivity.

To experimentally characterize the LEAP device's performance, the inventors placed the array in a three-electrode electrochemical cell containing saline buffer and applied controlled square-wave voltage steps using a potentiostat. The working electrode (400), is the gold contact with the ProDOT layer (130), was alternated between 0 mV and −100 mV to mimic the physiologically relevant bioelectric potential range generated by neurons. The setup included a platinum (Pt) counter electrode (420), an Ag/AgCl reference electrode (410). Throughout the voltage cycling, we continuously monitored changes in optical reflectance (320) from the LEAP device.

This experiment enabled calibration of the optical response across different geometries, yielding key performance metrics such as sensitivity, detection limit, and dynamic range. As expected, structures corresponding to the dark band exhibited the largest reflectance changes, confirming a strong correlation between low baseline reflection (high field confinement) and enhanced optical sensitivity.

After applying a square-wave voltage, different nanostructures exhibited distinct levels of reflection modulation, as shown in FIG. 4D. To quantify the optical response, the inventors normalized the reflectance change (FIG. 4D) by the baseline reflectance (FIG. 4C), yielding the percentage reflectance change for each geometry (FIG. 4F). A larger ΔR/R indicates a stronger optical response, enabling higher sensitivity through increased light intensity.

Critically, these experimental results closely align with simulations. The simulated reflection dips (FIG. 4G) strongly correlate with sensitivity trends (FIG. 4H)., because resonance wavelength shifts near the steep slope of the reflection dip produce the largest modulation. This strong agreement between experiment and simulation validates the LEAP sensing mechanism, confirming that near-field confinement and careful resonance tuning directly enhance voltage sensitivity.

### Large-Scale Optical Imaging of Neural and Cardiac Activity

In an exemplary embodiment, the large-scale, label-free optical imaging capabilities of LEAP is demonstrated by recording dynamic electrical activity from both cultured cardiomyocytes and acute brain slices (210). In each application, LEAP captures the spatial and temporal distribution of action potentials across multiple locations simultaneously, enabling high-throughput functional mapping at single-cell resolution.

To record cardiomyocyte activity, human induced pluripotent stem cell-derived cardiomyocytes (210) (hiPSC-CMs) were cultured directly on polymer-coated LEAP devices. Substrates were treated with adhesion-promoting layers followed by Matrigel coating to ensure robust cell attachment. Following replating, hiPSC-CMs exhibited rhythmic spontaneous beating, allowing direct optical recording of coordinated action potentials across the monolayer.

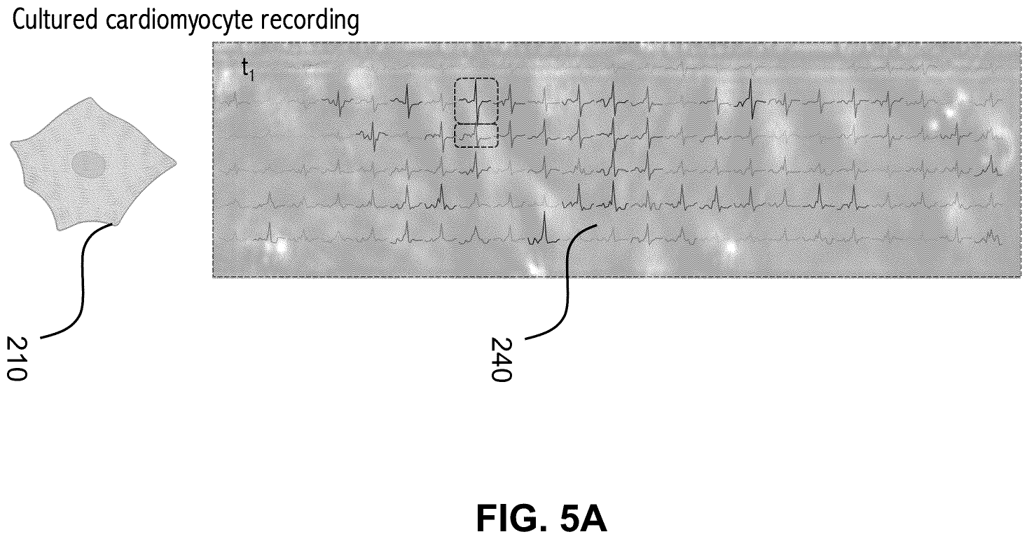

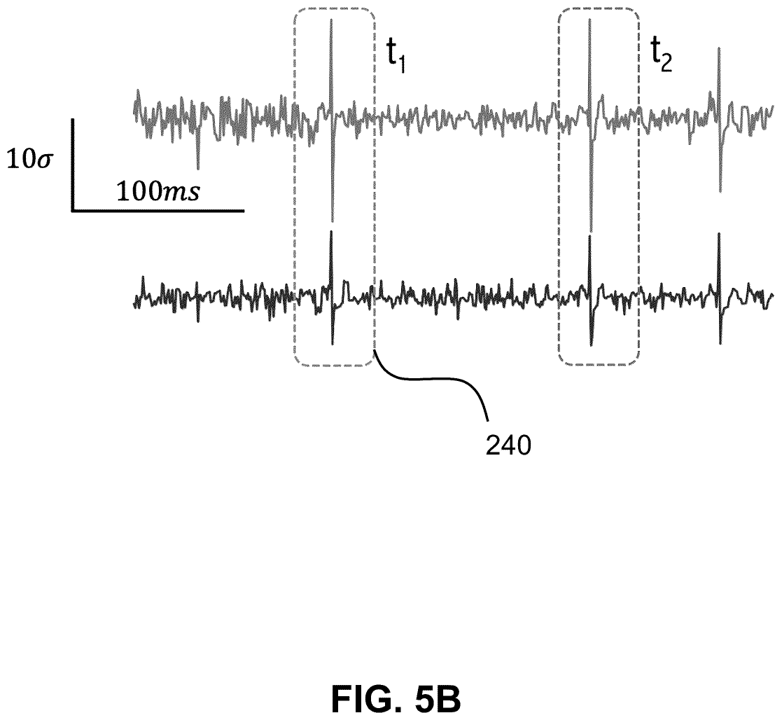

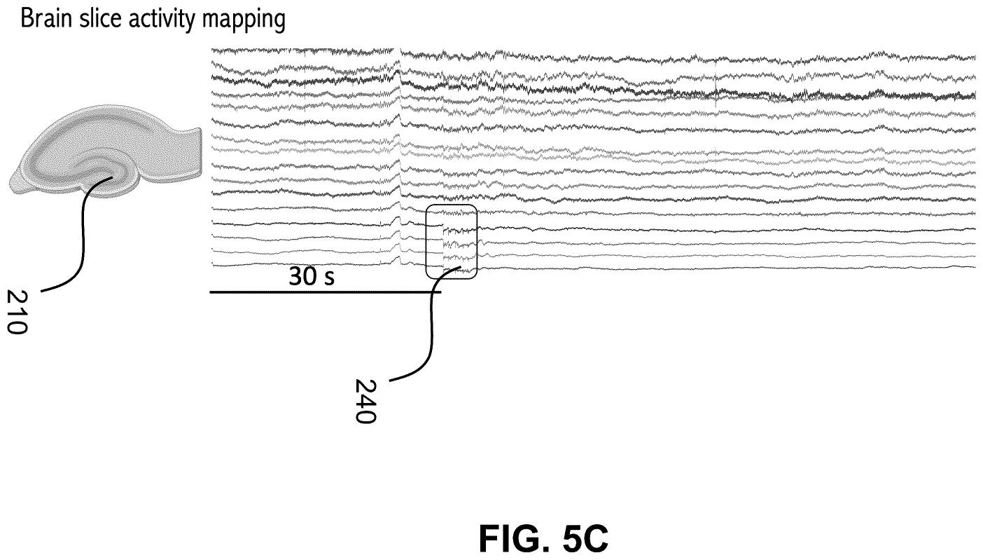

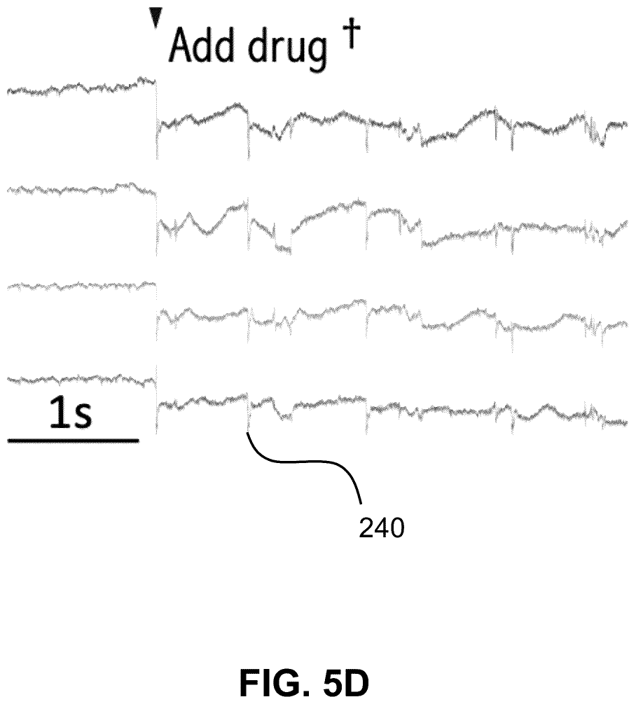

Raw video recordings achieved high spatial resolution (1 megapixel per 0.1 mm², ˜300 nm per pixel) and fast temporal resolution (4000 frames per second, or 0.25 ms per frame). Because 300 nm per pixel oversamples the biological signals, averaged pixel intensities were achieved to improve signal-to-noise ratio. Spike waveform maps are reconstructed at a 10 μm pitch overlaid on the original optical images, revealing that the recorded electrical activity closely correlates with the underlying cell topography, as illustrated in FIG. 5A. Traces extracted from two representative locations exhibit spike waveforms resembling extracellular action potentials measured with traditional multielectrode arrays, even without extensive spike sorting, as illustrated in FIG. 5B.

To extend LEAP's application to more complex tissue architectures, spontaneous neural activity was recorded from acute mouse brain slices (210). Mice were euthanized following institutional animal care protocols, and brains were rapidly extracted into ice-cold, oxygenated slicing buffer. Coronal slices (300 μm thick) were prepared using a vibratome under chilled conditions and immediately transferred onto polymer-coated LEAP devices using a fine brush, without additional surface treatments. Slices were maintained at room temperature in oxygenated artificial cerebrospinal fluid (ACSF) (200) during recordings. This minimally invasive preparation preserved native tissue architecture while enabling direct electrochemical coupling to the sensing layer.

After applying a bandpass filter to remove slow baseline fluctuations, we detected single-unit action potentials from multiple locations across the brain slice, as illustrated in FIG. 5C. Using advanced computational analysis—such as spike waveform clustering, inter-spike interval histograms, and dimensionality reduction via principal component analysis (PCA) and Uniform Manifold Approximation and Projection (UMAP)—high-resolution functional maps of neural activity were generated. LEAP enables electrophysiological imaging with both high spatial and temporal precision, allowing visualization of action potential propagation across complex tissue environments at unprecedented scales.

To validate the biological origin of the recorded spikes, the inventors pharmacologically modulated neural activity. Application of 6-cyano-7-nitroquinoxaline-2,3-dione (CNQX), an AMPAR antagonist, and tetrodotoxin (TTX), a voltage-gated sodium channel blocker, led to a marked reduction in firing rates recorded by LEAP. In contrast, treatment such as KCl, dopamine, or picrotoxin (a GABA_A receptor antagonist) increased firing rates, consistent with enhanced excitability, as illustrated in FIG. 5D. These results confirm that the optical signals captured by LEAP faithfully reflect underlying bioelectrical activity.

Together, these findings establish LEAP as a powerful platform for large-scale, label-free recording of action potentials, offering applications across basic neuroscience, cardiac physiology, pharmacological screening, and beyond.

### Fabrication of Single-Layer LEAP Devices

Quartz substrates were sequentially cleaned with acetone and isopropanol, followed by nitrogen drying, UV-ozone cleaning, and a dehydration bake. A thin layer of electron beam resist (CSAR 62) was spin-coated to a thickness of approximately 210 nm and baked at 180° C. for 1 minute. Nanoscale grating patterns were defined using electron beam lithography (EBL) and developed in xylene. A thin titanium (Ti) adhesion layer (˜1 nm) followed by a gold (Au) layer (˜30-100 nm) was deposited by electron beam evaporation at normal incidence. Liftoff was performed by soaking the samples in acetone overnight to produce clean metasurface structures. This process yielded single-layer metasurfaces that support surface plasmon resonances for enhanced optical sensitivity.

### Fabrication of Dual-Layer LEAP Devices

For dual-layer metasurfaces, after the above substrate cleaning and resist coating, grating patterns were defined by EBL and transferred into the quartz substrate using inductively coupled plasma (ICP) etching with CHF₃/O₂ chemistry. This etch produced trenches with vertical sidewalls approximately 50-150 nm deep. After resist removal, gold (˜30-100 nm) was directionally deposited by electron beam evaporation, coating both the top surface and the trench floors while maintaining electrical isolation between layers. This dual-layer structure, featuring independently confined Au layers at the top and trench bottom, enabled strong field confinement and enhanced voltage sensitivity via independent plasmonic oscillations. Careful optimization of etching and deposition minimized unwanted sidewall coverage, preserving optical performance. Alternatively, trenches can be fabricated by ion milling after an initial Au deposition, followed by a second Au deposition, offering a complementary route to achieving isolated double-layer metasurfaces.

### Operation of LEAP Devices Using Normal-Incidence Illumination and Optical Recording Setup

LEAP devices were operated under normal-incidence illumination to record optical signals corresponding to neuronal or cardiac electrical activity. Owing to the robust nanophotonic resonator design, which maintains strong optical response across a range of wavelengths and incident angles, LEAP uniquely enables high-sensitivity measurements without requiring oblique illumination or dark-field scattering geometries, unlike many prior techniques. This flexibility allows LEAP to operate with standard broadband light sources, such as LEDs, extending beyond expensive laser or super-luminescent diode (SLED) sources, without compromising device performance.

In a typical setup, collimated LED or laser light is focused into the back focal plane (BFP) of a microscope objective, passing through a beam splitter to ensure normal-incidence, wide-field illumination onto the LEAP metasurface. The nanophotonic metasurface efficiently couples and confines the incident light within the polymer layer, where voltage-induced refractive index changes from biological activity modulate the reflected light. The modulated reflected light retraces the optical path, passing back through the objective, is redirected by the beam splitter, and is relayed via a tube lens onto a high-speed camera for video acquisition. This setup leverages the engineered strong reflection modulation of the LEAP platform and is fully compatible with standard upright or inverted microscope architectures, providing a universal, cost-effective, and easily deployable imaging system for large-scale, label-free electrophysiological recording.

### hiPSC-Cardiomyocyte Culture and Preparation on LEAP Devices

Human induced pluripotent stem cell-derived cardiomyocytes (hiPSC-CMs) were cultured following an adapted protocol from the Joseph Wu laboratory. Cells were maintained in RPMI 1640 medium (Gibco, REF 11875-093) supplemented with B-27 (Gibco, REF 17504-044) at a 50:1 ratio. Culture medium was refreshed every 2-3 days. Before seeding cells, LEAP devices were prepared with a multi-step surface treatment. Substrates were first coated with 1 mg/mL poly-L-lysine (PLL) (in PBS) for 30 minutes at room temperature to promote initial adhesion. After three washes with PBS, devices were treated with 0.5% glutaraldehyde (in PBS) for 15 minutes to cross-link the PLL layer, followed by three additional PBS washes. Finally, devices were coated with Matrigel (Corning, REF 356231, diluted 1:200 in DMEM/F12) and incubated at 37° C. for at least 1 hour to provide an extracellular matrix environment.

For cell detachment and replating, the hiPSC-CM monolayers were gently washed with PBS without Ca²⁺/Mg²⁺ and incubated with TryPLE Select (Gibco, REF A12177-01) for 5-6 minutes at 37° C. Detached cells were neutralized by adding maintenance medium supplemented with 10% KnockOut Serum Replacement (KSR) (Gibco, REF A31815-01), collected by centrifugation (200 g for 3 minutes), and resuspended in fresh medium.

Cells were seeded onto the prepared LEAP devices at appropriate densities and incubated at 37° C. in a humidified 5% CO₂ atmosphere. After 24 hours, cell attachment was confirmed via light microscopy, and culture medium was switched back to RPMI-B27 maintenance conditions.

What is claimed is:

1. A nano-photonic chip for optical readout of biological activity, comprising:
(a) an optical substrate transparent for incoming light;
(b) a conductive path layer on top of the optical substrate;
(c) an electrochromic polymer layer on top of the conductive path layer,
wherein the electrochromic polymer layer is capable of changing optical properties of the electrochromic polymer layer in response to a biological activity,
wherein the electrochromic polymer layer has an optical absorption peak in a range of 400 nm to 700 nm, and
wherein optical absorption perturbation changes a refractive index of the electrochromic polymer layer; and
(d) a nearfield optical resonator formed between the conductive path layer and the electrochromic polymer layer, wherein the incoming light transmitted through the optical substrate is coupled by the conductive path layer into the nearfield optical resonator, wherein nearfield confinement enhances absorption in the electrochromic polymer layer, and wherein the changes in refractive index encode the biological activity and modulate the optical properties of outgoing reflected or transmitted light for optical readout therewith facilitating the optical readout of the biological activity.

2. The nano-photonic chip as set forth in claim 1, wherein the conductive path layer is a metal layer.

3. The nano-photonic chip as set forth in claim 2, wherein the metal layer is a nanopatterned metal grating, the metal layer is a metallic metasurface with a two-dimensional periodicity, the metal layer is a metal grating with a dual-layer nanopatterned grating structure having a unit cell, such that the combined structure exhibits multiple photonic modes with differing symmetry, or is a metal grating with a dual-layer nanopatterned grating structure, wherein each layer containing at least one grating elements of differing geometry within a unit cell.

4. The nano-photonic chip as set forth in claim 1, wherein the electrochromic polymer layer comprises a polymer backbone that supports a dominant π-π optical transition, and hydrophilic side chains that facilitate ionic interaction with a surrounding medium.

5. The nano-photonic chip as set forth in claim 1, wherein the electrochromic polymer layer exhibits a dominant π-π optical absorption peak between 500 nm and 650 nm, corresponding to its neutral-state transition prior to polaron formation.

6. The nano-photonic chip as set forth in claim 1, wherein the near-field resonator is selected from the group consisting of a plasmonic resonator, lattice resonator, a guided-mode resonator, a Fabry-Pérot resonator, and a gap-mode plasmonic resonator.

7. The nano-photonic chip as set forth in claim 1, wherein the biological activity comprises electrical, electrochemical activity or a combination thereof.

8. The nano-photonic chip as set forth in claim 1, further comprising a conductive path that includes: (i) an ionic solution penetrating the electrochromic polymer layer to form a local ionic interface with the polymer backbone, and (ii) a connection to a metal layer positioned outside a region of primary refractive modulation, the metal layer maintained near the solution's equilibrium potential, and functioning as a charge reservoir to enable fast electrochemical modulation of the electrochromic polymer layer.

9. The nano-photonic chip as set forth in claim 8, wherein the electrochromic polymer layer has hydrophilic side chains capable of ionic flow in response to the biological activity to charge a capacitor formed by the backbone of the conductive polymer layer and the ionic solution.

10. The nano-photonic chip as set forth in claim 1, wherein the incoming light is incident approximately normal to the optical substrate and outgoing light is reflected approximately normal to the optical substrate.

11. A nano-photonic chip for optical readout of biological activity, comprising:
(a) an optical substrate transparent for incoming light;
(b) a metal metasurface on top of the optical substrate for coupling the incoming light, wherein the metal metasurface comprises multiple patterned layers, each having one or more grating sub-elements with different geometries within a unit cell, configured as either a one-dimensional metasurface grating or a two-dimensional metasurface array;
(c) an electrochromic polymer layer on top of the metal metasurface,
wherein the electrochromic polymer layer is capable of changing optical properties of the electrochromic polymer layer in response to a biological activity,
wherein the electrochromic polymer layer has an optical absorption peak in a range of 400 nm to 700 nm, and
wherein optical absorption perturbation changes a refractive index of the conductive polymer; and
(d) a nearfield optical resonator formed between the metal metasurface and the electrochromic polymer layer, wherein the incoming light transmitted through the optical substrate is coupled by the metal metasurface into the nearfield optical resonator, wherein the changes in refractive index encode the biological activity and therewith facilitating optical readout of the biological activity.

12. The nano-photonic chip as set forth in claim 11, wherein the electrochromic polymer layer comprises a polymer backbone that supports a dominant π-π optical transition, and hydrophilic side chains that facilitate ionic interaction with a surrounding medium.

13. The nano-photonic chip as set forth in claim 11, wherein the electrochromic polymer layer exhibits a dominant π-π optical absorption peak between 500 nm and 650 nm, corresponding to its neutral-state transition prior to polaron formation.

14. The nano-photonic chip as set forth in claim 11, wherein the near-field resonator is selected from the group consisting of a plasmonic resonator, lattice resonator, a guided-mode resonator, a Fabry-Pérot resonator, and a gap-mode plasmonic resonator.

15. The nano-photonic chip as set forth in claim 11, wherein the biological activity comprises electrical, electrochemical activity or a combination thereof.

16. The nano-photonic chip as set forth in claim 11, further comprising a conductive path that includes: (i) an ionic solution penetrating the electrochromic polymer layer to form a local ionic interface with the polymer backbone, and (ii) a connection to a metal layer positioned outside the region of primary refractive modulation, the metal layer maintained near a solution's equilibrium potential, and functioning as a charge reservoir to enable fast electrochemical modulation of the electrochromic polymer layer.

17. The nano-photonic chip as set forth in claim 16, wherein the electrochromic polymer layer has hydrophilic side chains capable of ionic flow in response to the biological activity to charge a capacitor formed by the backbone of the conductive polymer layer and the ionic solution.

18. The nano-photonic chip as set forth in claim 11, wherein the incoming light is incident approximately normal to the optical substrate.
